(19) United States
(12) Patent Application Publication
(10) Pub. No.: US 2023/0295597 A1
(43) Pub. Date: Sep. 21, 2023

(54) A DISINTEGRIN AND METALLOPROTEINASE WITH A THROMBOSPONDIN TYPE I MOTIF, MEMBER 13 (ADAMTS-13) MUTANTS, COMPOSITIONS AND THERAPEUTIC METHODS THEREOF

(71) Applicant: HADASIT MEDICAL RESEARCH SERVICES & DEVELOPMENT LIMITED, Jerusalem (IL)

(72) Inventors: Abd Alrauf HIJAZI, D.N. Shimshon (IL); Suhair Abd Eljawad ABDEEN, Jerusalem (IL)

(21) Appl. No.: 18/041,493

(22) PCT Filed: Aug. 12, 2021
(86) PCT No.: PCT/IL2021/050988

§ 371 Date: Feb. 13, 2023

Related U.S. Application Data
(60) Provisional application No. 63/065,204, filed on Aug. 13, 2020.

(51) Int. Cl. C12N 9/64; C07K 14/46; A61K 38/17; A61K 38/48; A61K 38/36; A61P 7/02

(52) U.S. Cl. CPC C12N 9/6489; C07K 14/46; A61K 38/1703; C12Y 304/24087; A61K 38/4886; C12Y 304/24068; A61K 38/36; A61P 7/02; C07K 2319/35

(57) ABSTRACT

The present invention relates to ADAMTS-13 mutants and/or variant/s that display resistance to tPA cleavage and/or inactivation. The present disclosure further provides fibrinolytic compounds, compositions, combined compositions and kits comprising the ADAMTS-13 mutants disclosed herein, as well as uses thereof for treating coagulation related disorders.

### FIELD OF THE INVENTION

The invention relates to coagulation modulators. More specifically, the invention relates to disintegrin and metalloproteinase with a thrombospondin type 1 motif, member 13 (ADAMTS-13) mutants and/or variant, compositions and uses thereof for treating coagulation related disorders.

### BACKGROUND ART

References considered to be relevant as background to the presently disclosed subject matter are listed below:
[1] Bao J, Xiao J, Mao Y, Zheng X L. Arterioscler Thromb Vasc Biol 2014, 34(2): 397-407, Carboxyl terminus of ADAMTS-13 directly inhibits platelet aggregation and ultra large von Willebrand factor string formation under flow in a free-thiol-dependent manner.
[2] Galbusera M, Noris M, Remuzzi G. Semin Thromb Hemost 2006 32(2): 81-89, Thrombotic thrombocytopenic purpura—then and now.
[3] Bongers T N, de Maat M P, van Goor M L, Bhagwanbali V, van Vliet H H, Gómez Garcia E B, Dippel D W, Leebeek F W. Stroke. 2006 November; 37(11):2672-7; High von Willebrand factor levels increase the risk of first ischemic stroke: influence of ADAMTS-13, inflammation, and genetic variability.
[4] Galbusera M, Noris M, Remuzzi G. Semin Thromb Hemost. 2006 March; 32(2):81-9. Review. Thrombotic thrombocytopenic purpura—then and now.
[5] Zhao B Q, Chauhan A K, Canault M, Patten I S, Yang J J, Dockal M, Scheiflinger F, Wagner D D. Blood. 2009 Oct. 8; 114(15):3329-34. von Willebrand factor-cleaving protease ADAMTS-13 reduces ischemic brain injury in experimental stroke.
[6] Zeng M, Chen Q, Liang W, He W, Zheng H, Huang C, Int J Chron Obstruct Pulmon Dis. 2017 Dec. 5; 12:3495-3501. Predictive value of ADAMTS-13 on concealed chronic renal failure in COPD patients.
[7] Denorme F, Langhauser F, Desender L, Vandenbulcke A, Rottensteiner H, Plaimauer B, François O, Andersson T, Deckmyn H, Scheiflinger F, Kleinschnitz C, Vanhoorelbeke K, De Meyer S F. Blood. 2016 May 12; 127(19):2337-45. doi: 10.1182/blood-2015-08-662650. Epub 2016 Feb. 29. ADAMTS-13-mediated thrombolysis of t-P A-resistant occlusions in ischemic stroke in mice.
[8] José A. Diaz, Angela E. Hawley, Christine M. Alvarado, Alexandra M. Berguer, Nichole K. Baker, Shirley K. Wrobleski, Thomas W. Wakefield, Benedict R. Lucchesi, and Daniel D. Myers, Jr. Thromb Haemost 2010; 104: 366-375, Thrombogenesis with continuous blood flow in the inferior vena cava: A novel mouse model.
[9] Denorme F, Langhauser F, Desender L, Vandenbulcke A, Rottensteiner H, Plaimauer B, François O, Andersson T, Deckmyn H, Scheiflinger F, Kleinschnitz C, Vanhoorelbeke K, De Meyer S F. Blood 2016 127:2337-2345, ADAMTS-13-mediated thrombolysis of t-P A-resistant occlusions in ischemic stroke in mice.
[10] Schuhmann M K, Gunreben I, Kleinschnitz C, Kraft P. Int J Mol Sci 2016 17(3), Int J Mol Sci. 2016; 17(3):298. Immunohistochemical Analysis of Cerebral Thrombi Retrieved by Mechanical Thrombectomy from Patients with Acute Ischemic Stroke.
[11] Miszta A, Pelkmans L, Lindhout T, Krishnamoorthy G, de Groot P G, Hemker C H, Heemskerk J W, Kelchtermans H, de Laat B. J Biol Chem 2014, 289(52): 35979-35986, Thrombin-dependent Incorporation of von Willebrand Factor into a Fibrin Network.
[12] Bhatia R, Hill M D, Shobha N, Menon B, Bal S, Kochar P, Watson T, Goyal M, Demchuk A M. Stroke 2010, 41(10): 2254-2258, Low rates of acute recanalization with intravenous recombinant tissue plasminogen activator in ischemic stroke: real-world experience and a call for action.
[13] Kleinschnitz C, De Meyer S F, Schwarz T, Austinat M, Vanhoorelbeke K, Nieswandt B, Deckmyn H, Stoll G. Blood 2009 113(15): 3600-3603, Deficiency of von Willebrand factor protects mice from ischemic stroke.
[14] Kleinschnitz C¹, De Meyer S F, Schwarz T, Austinat M, Vanhoorelbeke K, Nieswandt B, Deckmyn H, Stoll G. Blood. 2009 Apr. 9; 113(15):3600-3. Deficiency of von Willebrand factor protects mice from ischemic stroke.
[15] Fujioka M, Hayakawa K, Mishima K, Kunizawa A, Irie K, Higuchi S, Nakano T, Muroi C, Fukushima H, Sugimoto M, Banno F, Kokame K, Miyata T, Fujiwara M, Okuchi K, Nishio K. Blood 2010 115(8): 1650-1653, ADAMTS-13 gene deletion aggravates ischemic brain damage: a possible neuroprotective role of ADAMTS-13 by ameliorating postischemic hypoperfusion.
[16] Sonneveld M A, de Maat M P, Portegies M L, Kavousi M, Hofman A, Turecek P L, Rottensteiner H, Scheiflinger F, Koudstaal P J, Ikram M A, Leebeek F W. Blood 2015 126(25): 2739-2746. Low ADAMTS-13 activity is associated with an increased risk of ischemic stroke.
[17] Zheng X L. Annu Rev Med 2015 66: 211-225, ADAMTS-13 and von Willebrand factor in thrombotic thrombocytopenic purpura.
[18] E P 2172544.
[19] Thomas M R, de Groot R, Scully M A, Crawley J T. EBioMedicine 2015 2, 942-952. Pathogenicity of Anti-ADAMTS-13 Autoantibodies in Acquired Thrombotic Thrombocytopenic Purpura.
[20] Denorme F, Langhauser F, Desender L, Vandenbulcke A, Rottensteiner H, Plaimauer B, François O, Andersson T, Deckmyn H, Scheiflinger F, Kleinschnitz C, Vanhoorelbeke K, De Meyer S F. Blood 2016 127:2337-2345. ADAMTS-13-mediated thrombolysis of t-PA-resistant occlusions in ischemic stroke in mice.
[21] Diaz J A, Hawley A E, Alvarado C M, Berguer A M, Baker N K, Wrobleski S K, Wakefield T W, Lucchesi B R, Myers D D Jr. Thromb Haemost 2010 104 366-375. Thrombogenesis with continuous blood flow in the inferior vena cava. A novel mouse model.
[22] Feys, H B, Vandeputte, N, Palla R, Peyvandi F, Peerlinck K, Deckmyn H, Lijnen H R and Vanhoorelbeke K. Jr. Thromb Haemost 2010 8: 2053-62. Inactivation of ADAMTS13 by plasmin as a potential cause of thrombotic thrombocytopenic purpura

Acknowledgement of the above references herein is not to be inferred as meaning that these are in any way relevant to the patentability of the presently disclosed subject matter.

### BACKGROUND OF THE INVENTION

Dysregulation of hemostasis is central to the pathogenesis of thrombotic disorders. Under physiologic conditions, fibrin formation and fibrinolysis are coordinately regulated both temporally and locally. The endothelium contributes to maintaining this balance, in part, by releasing von Willebrand Factor (vWF) multimers from specialized granules in response to endothelial stress or damage, and by that initiates thrombus formation. vWF is a large and heterogeneous, multidomain adhesive glycoprotein (GP) that is essential for normal hemostatic function. It is synthesized in endothelial cells as a monomer that dimerizes in the endoplasmic reticulum through a C-terminal disulfide bond. Heterogeneous vWF multimers are stored within Weibel-Palade bodies, from which they can be released constitutively and upon demand.

vWF multimers undergo shear-induced conformational changes forming ultralarge multimers (ULvWF) along the luminal surface of the damaged endothelium that promote platelet adhesion that is followed by fibrin accumulation and ends by thrombus formation. ULvWF activity is constrained by proteolytic inactivation by a disintegrin and metalloproteinase with a thrombospondin type 1 motif, member 13 (ADAMTS-13) [1, 2]. ADAMTS-13 is deficient in patients with congenital or acquired autoimmune thrombotic thrombocytopenic purpura (TTP) who develop disseminated microvascular thrombosis and have increased tendency to develop Myocardial Infarction (MI), acute ischemic stroke (AIS) [3-7] and Deep vein thrombosis (DVT), where it is considered as potential therapeutic target [8]. Most TTP patients have acquired anti-ADAMTS-13 autoantibodies that inhibit enzyme function and/or clear it from the circulation. In one of these patients, inactivation of ADAMTS-13 by excessive proteolysis mediated via plasmin was reported [22].

ADAMTS-13 is a 190 kDa glycosylated metalloproteinase produced by endothelial and hepatic stellate cells. Cleavage of ULvWF multimers by ADAMTS-13 reduces platelet adhesion and down-regulates thrombus formation. In vivo studies using a DVT model in mice, show that the formed clots contain substantial amount of vWF [8]. Likewise, recent studies show that human cerebrovascular thrombi contain variable amounts of vWF [9, 10] and that multimers of vWF integrate into polymerized fibrin [11] which correlate with resistance to tPA [12] but not to ADAMTS-13 [9].

Previous experimental and clinical studies suggest that levels of vWF and ADAMTS-13 are linked to the risk of AIS, DVT and pulmonary emboli (PE), and that ADAMTS-13 also protects the brain from reperfusion injury in AIS [13-17].

EP2172544 [18] discloses several ADAMTS-13 mutants exhibiting enhanced or reduced catalytic activity, created to provide ADAMTS-13 variants that display reduced immunogenicity. Specifically, this publication teaches that such mutants may be produced by substituting a charged amino acid such as arginine (e.g., R312 or R326 or R370), Lysine (e.g., K318 or K608), glutamic acid (E), aspartic acid (D)) in the disintegrin-like domain, the cysteine-rich domain or the spacer domain, with a different amino acid, especially an uncharged amino acid. Accordingly, this publication teaches that charged amino acid residues of ADAMTS-13 such as arginine or lysine, must be replaced by uncharged amino acid residues, such as alanine.

Thrombotic and Thromboembolic events such as venous thromboembolism (VTE) including deep venous thrombosis (DVT), pulmonary emboli (PE) and acute ischemic stroke (AIS), are a major cause of death and disabilities. There is currently no effective medical treatment of such conditions, and therefore an unmet need exists for development of effective modulators for thrombotic processes.

### SUMMARY OF THE INVENTION

In a first aspect, the invention relates to a mutant of a disintegrin and metalloproteinase with a thrombospondin type 1 motif, member 13 (ADAMTS-13) that carries at least one mutation. The ADAMTS-13 mutant disclosed herein displays resistance and/or reduced sensitivity to cleavage and/or inactivation by at least one tissue plasminogen activator (tPA), or any mutant or variant thereof. In some embodiments, at least one of the mutations of the mutants of the invention may substitute the Arginine in position 312 (Arg312) or in any amino acid residue adjacent to the Arg312 of the wild type ADAMTS-13 with a charged amino acid residue, or any truncated variant thereof. It should be understood that residue 312, as indicated herein refers to the amino acid sequence of the wild type ADAMTS-13, that may comprise the amino acid sequence as denoted by SEQ ID NO: 2, or any variants or derivatives thereof.

In yet some other aspect, the invention relates to a composition comprising an effective amount of at least one ADAMTS-13 mutant, or any truncated variant thereof. In some embodiments, the mutant may carry a mutation and displays resistance and/or reduced sensitivity to cleavage and/or inactivation by at least one tPA, or any mutant or variant thereof. In some embodiments, the mutant carries at least one mutation that substitute the Arg312 residue and/or any amino acid residue adjacent to the Arg312 of the wild type ADAMTS-13 with a charged amino acid residue. It should be noted that the wild type ADAMTS-13 comprises the amino acid sequence as denoted by SEQ ID NO: 2, and therefore the position 312, refers to SEQ ID NO: 2. In some embodiments, the composition may optionally further comprise at least one pharmaceutically acceptable carrier, diluent, excipient and/or additive.

In another aspect, the invention relates to a combined composition comprising a combination of at least one ADAMTS-13 mutant and/or any variant thereof, and at least one tPA or any functional fragments or variants thereof. In some embodiments, the mutant may carry at least one mutation and displays resistance and/or reduced sensitivity to cleavage and/or inactivation by at least one tPA, or any mutant or variant thereof. In some embodiments, at least one of the mutations of the mutants may substitute the Arg312 residue or any amino acid residue adjacent to the Arg312 of the wild type ADAMTS-13 with a charged amino acid residue. It should be noted that the wild type ADAMTS-13 may comprise the amino acid sequence as denoted by SEQ ID NO: 2, or any variants or derivatives thereof. Still further, in some embodiments, the composition may optionally further comprise at least one pharmaceutically acceptable carrier, diluent, excipient and/or additive.

In a further aspect, the invention relates to a method for the treatment, amelioration, inhibition or prophylaxis of a disease, disorder, or condition associated with coagulation in a subject in need thereof. In some embodiments, the method may comprise the step of administering to the subject a therapeutically effective amount of at least one ADAMTS-13 mutant, any truncated variant thereof, or any composition or combined composition comprising the mutant of the invention. In certain embodiments, the mutants of the invention may carry at least one mutation and display resistance and/or reduced sensitivity to cleavage and/or inactivation by at least one tPA, or any mutant or variant thereof. In some embodiments, at least one of the mutations of the disclosed mutants may substitute the Arg312 residue or any amino acid residue adjacent to the Arg312 of the wild type ADAMTS-13 with a charged amino acid residue. It should be noted that the wild type ADAMTS-13 may comprise the amino acid sequence as denoted by SEQ ID NO: 2.

In another aspect, the invention relates to a method for the treatment, amelioration, inhibition or prophylaxis of a disease, disorder, or condition associated with coagulation in a subject in need thereof, comprising the step of administering to a subject treated with at least one tPA, or any mutant or variant thereof, a therapeutically effective amount of at least one ADAMTS-13 mutant, and/or any variant thereof or any composition comprising the mutant. In some embodiments, the mutant may carry at least one mutation and display resistance and/or reduced sensitivity to cleavage and/or inactivation by at least one tPA, or any mutant or variant thereof. In some embodiments, the mutant carries at least one mutation that substitutes the Arg312 residue or any amino acid residue adjacent to said Arg312 of the wild type ADAMTS-13 with a charged amino acid residue. The wild type ADAMTS-13 may comprise the amino acid sequence as denoted by SEQ ID NO: 2, or any variants or derivatives thereof.

In yet another aspect, the invention relates to a kit comprising:

First (a), at least one ADAMTS-13 mutant, and/or any variant thereof, or any composition thereof. In certain embodiment, the mutant may carry at least one mutation, and display resistance and/or reduced sensitivity to cleavage and/or inactivation by at least one tPA, or any mutant or variant thereof. In some embodiments, the mutant carries at least one mutation that substitutes the Arg312 residue and/or any amino acid residue adjacent to the Arg312 of the wild type ADAMTS-13 with a charged amino acid residue. The said wild type ADAMTS-13 may comprise the amino acid sequence as denoted by SEQ ID NO: 2. The second component (b), may comprise at least one tPA or any functional fragments or variants thereof, or any composition thereof.

In a further aspect, the invention relates to at least one ADAMTS-13 mutant, and/or any variant thereof or any composition comprising the mutant, for use in a method of treatment, amelioration, inhibition or prophylaxis of a disease, disorder, or condition associated with coagulation in a subject in need thereof. In some embodiments, the mutant may carry at least one mutation and displays resistance and/or reduced sensitivity to cleavage and/or inactivation by at least one tPA, or any mutant or variant thereof. In some embodiments, the mutant carries at least one mutation that substitutes the Arg312 residue or any amino acid residue adjacent to the Arg312 of the wild type ADAMTS-13 with a charged amino acid residue. It should be noted that the wild type ADAMTS-13 comprises the amino acid sequence as denoted by SEQ ID NO: 2.

The invention further provides a therapeutically effective amount of at least one ADAMTS-13 mutant and/or any variant thereof or any composition comprising the mutant for use in a method for the treatment, amelioration, inhibition or prophylaxis of a disease, disorder, or condition associated with coagulation in a subject in need thereof. It should be noted that the subject is a subject being treated with at least one tPA, or any mutant or variant thereof. In yet some further specific embodiments, the mutant carries at least one mutation, and displays resistance and/or reduced sensitivity to cleavage and/or inactivation by at least one tPA, or any mutant or variant thereof. In more specific embodiments, the mutation carried by the disclosed mutant and/or variant substitutes the Arg312 residue or any amino acid residue adjacent to said Arg312 of the wild type ADAMTS-13 with a charged amino acid residue.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-C: tPA cleaves native ADAMTS-13 in cells and in human plasma

FIG. 1A: Western blot analysis of lysates from fetal liver cells (A549 cells) incubated in saline (Lysates), with tPA (100 nM) (tPA) or tPA (100 nM) and aprotinin (1 μM) (tPA+Ap) for 2 hours. Blot was incubated with monoclonal anti-ADAMTS-13 antibody EPR6132 (Abcam).

FIG. 1B: Western blot analysis of human plasma incubated in saline (Plasma), with tPA (100 nM) (tPA) or tPA (100 nM) and aprotinin (1 μM) (tPA+Ap) for 2 hours. Blot was incubated with monoclonal anti-ADAMTS-13 antibody EPR6132 (Abcam).

FIG. 1C: Graph showing percent of activity of ADAMTS-13 in A549 cell lysates and human plasma. ADAMTS-13 activity was determined by technozym ADAMTS-13 activity (Technoclone).

FIG. 2: tPA inactivates ADAMTS-13 in vivo

Graph showing percent of activity of ADAMTS-13 and vWF in plasma from WT and tPA−/− mice. ADAMTS-13 activity was determined by technozym ADAMTS-13 activity (Technoclone), vWF antigen (Ag) was measured by Von willebrand factor Ag hemosil reagent (instrumentation laboratory) and vWF Ristocetin cofactor hemosil reagent (instrumentation laboratory).

FIG. 3A-3B: ADAMTS-13 structure

FIG. 3A: Schematic representation of human ADAMTS-13, a multidomain protein with Metalloprotease domain (MP), Disinterring-like domain (Disin), 8 Thrombospondin type-1 domains (1-8), Cysteine-rich domain (Cys), Spacer domain (S) and 2 CUB domains.

FIG. 3B: Schematic representation of truncated ADAMTS-13 variant (MDTCS) i.e. trADAMTS-13 that contains MP domain and is catalytically active.

FIG. 4A-4D: Characterization of the truncated ADAMTS-13 variant FIG. 4A: SDS-PAGE of the truncated ADAMTS-13 variant (trADAMTS-13) (Lanes 2-3).

FIG. 4B: Graph showing activity of ADAMTS-13 WT and trADAMTS-13 without or with preincubation with tPA using a technozym kit to measure ADAMTS-13 activity (Technoclone).

FIG. 4C: Western blot analysis of truncated recombinant human ADAMTS-13 (trADAMTS-13) (500 nM) incubated with tPA (100 nM) for 0, 1, 2, 4 and 8 hours using monoclonal anti-ADAMTS-13 antibody (Abcam).

FIG. 4D: Western blot analysis using anti-vWF antibody (Abcam) of vWF multimers in human plasma incubated in the presence or absence of trADAMTS-13 (300 nM) alone (for 3 hours) or with preincubation with tPA (100 nM, for 2 hours) at 37° C.

FIG. 5: Delineation of the trADAMTS-13 cleavage site SDS-PAGE gel (10%) of trADAMTS-13 incubated without or with tPA for 4 hours. Individual bands were excised and their amino acid sequences were determined.

FIG. 6A-6B: Mutations of the trADAMTS-13 cleavage site and generation of tPA resistant variants

FIG. 6A: SDS-PAGE of the truncated ADAMTS-13 variants: trADAMTS-13 WT (Lane 1), trADAMTS-13^{R312K} (Lane 2), trADAMTS-13^{R312A} (Lane 3), trADAMTS-13^{V313A}(Lane 4) and trADAMTS-13^{V313D} (Lane 5).

FIG. 6B: Graph showing catalytic activity of the truncated ADAMTS-13 variants on vWF.

FIG. 7A-7B: trADAMTS-13^{R312K} is resistant to tPA cleavage FIG. 7A: Western blot analysis of trADAMTS-13 WT and trADAMTS-13^{R312K} incubated with tPA for the indicated periods of times. Blot was incubated with monoclonal anti-ADAMTS-13 antibody (Abcam).

FIG. 7B: Graph showing percentage of ADAMTS-13 proteolytic activity on vWF of both ADAMTS-13 truncated proteins, trADAMTS-13 WT (Black) and trADAMTS-13^{R312K}(Gray) determined after the indicated periods of time of incubation with tPA.

FIG. 8: trADAMTS-13 variants are resistant to cleavage by tPA Graph showing residual activity of the trADAMTS-13 variants incubated with or without tPA for 6 hours. Proteolytic activity on vWF of the variants was determined after the incubation. Residual activity shows the activity obtained in presence of tPA in comparison to the activity in the absence of tPA.

FIG. 9A-9B: Effect of autoantibodies from TTP plasma on trADAMTS-13 variants activity

FIG. 9A: Graph showing residual activity of trADAMTS-13 variants incubated for 2 hours with plasma from patients with acquired ADAMTS-13 deficiency. Proteolytic activity on vWF of the variants was determined after the incubation. Residual activity shows the activity obtained after incubation with TTP plasma in comparison to that detected before the incubation.

FIG. 9B: Graph showing activity of trADAMTS-13 WT and trADAMTS-13^{R312K} incubated for 2 hours or 24 hours with plasma from patients with acquired ADAMTS-13 deficiency. Proteolytic activity on vWF of the variants was determined before and after the incubation with plasma. The activity obtained after incubation with TTP plasma in comparison to that detected before the incubation (mean±SD, p<0.05, n=5 each group).

FIG. 10: Thrombolytic effect of trADAMTS-13 variants on VWF-rich thrombus Graph showing time to reperfusion following no treatment (control), treatment with tPA (tPA), treatment with tPA and trADAMTS-13^{WT} (tPA+trADAMTS-13^{WT}) treatment with tPA and trADAMTS-13^{R312K} (tPA+trADAMTS-13^{R312K}), treatment with trADAMTS-13^{WT} or treatment with the mutant alone trADAMTS-13^{R312K}. Five minutes after occlusion, mice were given intravenous injection of tPA (0.5 mg/kg) alone or together with trADAMTS-13^{WT} or trADAMTS-13^{R312K} (5 mg/kg each), and blood reperfusion was monitored for 120 minutes. In the control group, no improvement was detected in blood flow even after 120 minutes of monitoring.

FIG. 11A-11C: In vivo anti-vWF activity of trADAMTS-13 variants FIG. 11A: Graph showing percentage of ADAMTS-13 activity in plasma of WT and tPA −/− mice injected IP with trADAMTS-13 WT or trADAMTS-13^{R312K} (1 mg, after two hours).

FIG. 11B: Graph showing concentration of vWF in plasma of WT and tPA −/− mice injected IP with trADAMTS-13 WT or trADAMTS-13^{R312K} (1 mg, after two hours).

FIG. 11C: Graph showing percentage of vWF activity in plasma of WT mice injected IP with trADAMTS-13 WT or trADAMTS-13^{R312K} (1 mg, after two hours).

FIG. 12A-12B: In vivo anti-thrombotic activity of trADAMTS-13 variants

FIG. 12A: Graph showing the weight of Thrombus (mg) in WT and tPA −/− mice after inducing venous clots formation using an IVC stasis model (Blood 2015 125(16): 2558-2567).

FIG. 12B: Graph showing the weight of Thrombus (mg) in WT mice administered with IP injection of (1 mg) trADAMTS-13 WT or trADAMTS-13^{R312K} one hour before inducing venous clots formation using an IVC stasis model (Blood 2015 125(16): 2558-2567).

FIG. 13: In vivo anti-coagulant activity of trADAMTS-13 variants Graph showing bleeding time (min) of WT mice administered with IP injection of (1 mg) trADAMTS-13 WT or trADAMTS-13^{R312K} two hours before inducing bleeding using the tail cut model bleeding. Tails were cut as in (Blood. 2019 Jan. 31; 133(5):481-493) and the bleeding times were determined as in (Blood. 2019 Jan. 31; 133(5):481-493).

FIG. 14A-14B: Soluble fibrin inhibits tPA-mediated inactivation of ADAMTS-13

FIG. 14A: Graph showing activity of the trADAMTS-13 incubated for 4 hours in the presence/absence of tPA (100 nM) and/or fibrin (2 μM) (mean±SD, n=3 each group).

FIG. 14B: Graph showing activity of full length ADAMTS-13 in serum from WT mice incubated for 4 hours in PBS (serum) or PBS containing tPA (100 nM) or tPA and fibrin (0.1 mg/ml) (mean±SD, n=3 each group).

### DETAILED DESCRIPTION OF THE INVENTION

Recent evidence indicates that ADAMTS-13 plays an important role in thrombotic and thromboembolic events [20, 21]. Currently, there is no effective medical treatment related conditions such as venous thromboembolism (VTE) including deep venous thrombosis (DVT) and pulmonary emboli (PE) and acute ischemic stroke (AIS).

In the present application, improved ADAMTS-13 variants were developed, specifically ADAMTS-13 exhibiting enhanced activity, which represent novel thrombolytic and anticoagulant agents.

Therefore, in a first aspect, the invention relates to a mutant and/or variant of a disintegrin and metalloproteinase with a thrombospondin type 1 motif, member 13 (ADAMTS-13) that carries at least one mutation. In yet some further embodiments, the mutants and/or variants disclosed herein display resistance and/or reduced sensitivity to cleavage and/or inactivation by at least one tissue plasminogen activator (tPA), or any mutant or variant thereof.

In some embodiments, the mutant and/or variant of the present disclosure carries at least one mutation. In some embodiments, at least one of the mutations in the mutants of the invention may substitute the Arginine in position 312 (Arg312) and/or in any amino acid residue adjacent to the Arg312 of the wild type ADAMTS-13 with a charged amino acid residue, or any truncated variant thereof. It should be understood that residue 312, as indicated herein refers to the amino acid sequence of the wild type ADAMTS-13, that may comprise the amino acid sequence as denoted by SEQ ID NO: 2, or any variants, homologues or derivatives thereof.

ADAMTS-13 (a disintegrin and metalloproteinase with a thrombospondin type 1 motif, member 13), also known as von Willebrand factor-cleaving protease (VWFCP), and also referred to herein as ADAMTS-13, ADAMTS13, and ADAMTS, is a zinc-containing metalloprotease enzyme that cleaves von Willebrand factor (vWf), a large protein involved in blood clotting. It is secreted into the blood and degrades large vWf multimers, decreasing their activity. Genomicaly, ADAMTS-13 shares many properties with the 19 member ADAMTS family, all of which are characterized by a protease domain (the part that performs the protein hydrolysis), an adjacent disintegrin domain and one or more thrombospondin domains. ADAMTS-13 in fact has eight thrombospondin domains. It has no hydrophobic transmembrane domain, and hence it is not anchored in the cell membrane. Human ADAMTS-13 is a multidomain protein with Metalloprotease domain (MP), Disinterring-like domain (Disin), 8 Thrombospondin type-1 domains (1-8), Cysteine-rich domain (Cys), Spacer domain (S) and 2 CUB domains. The metalloprotease domain contains the catalytic site of ADAMTS-13 that cleaves vWF. The ADAMTS-13 gene maps to the ninth chromosome (9q34) and its accession number is NM_139025.4. It encodes for the amino acid sequence having the accession number NP_620594.1.

In some embodiments, the human wild type ADAMTS-13 protein may comprise the amino acid sequence as denoted by SEQ ID NO: 2, or any variants, homologues or derivatives thereof. In some further embodiments, the wild type ADAMTS-13 protein may be encoded by a nucleic acid sequence comprising the nucleic acid sequence as denoted by SEQ ID NO: 1, or any variants, homologues or derivatives thereof.

The present invention provides mutants of the ADAMTS-13 protein. The term “mutant” or “mutant protein,” as used herein, refers to a protein product encoded by a gene with mutation. Specifically, it should be appreciated that “ADAMTS-13 mutant” as used herein includes a mutated native and recombinant mutated ADAMTS-13 protein, as well as modified forms of ADAMTS-13 that display increased activity in comparison with the enzymatic activity displayed by the wild type ADAMTS-13. More specifically, the term “mutation” as herein defined refers to a change in the nucleotide sequence of the genome of an organism, for example, in the nucleic acid sequence encoding the ADAMS-13 protein. Mutations may or may not produce observable (phenotypic) changes in the characteristics of the encoded protein. However, in some embodiments, the mutants disclosed herein display a modified sensitivity to proteolytic cleavage and/or inactivation. Mutation can result in several different types of change in the DNA sequence; these changes may have no effect, alter the product of a gene, or prevent the gene from functioning properly or completely. There are generally three types of mutations, namely single base substitutions, insertions and deletions and mutations defined as “chromosomal mutations”. The term “single base substitutions” as herein defined refers to a single nucleotide base which is replaced by another. These single base changes are also called point mutations. There are two types of base substitutions, namely, “transition” and “transversion”. When a purine base (i.e., Adenosine or Thymine) replaces a purine base or a pyrimidine base (Cytosine, Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows. In missense mutations the new base alters a codon, resulting in a different amino acid being incorporated into the protein chain.

In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly.

Mutation may also arise from insertions of nucleic acids into the DNA or from duplication or deletions of nucleic acids therefrom. As herein defined, the term “insertions and deletions” refers to extra base pairs that are added or deleted from an encoding nucleic acid sequence, respectively. Insertions and deletions of one or two bases or multiples of one or two bases cause, inter alia, frame shift mutations (i.e., these mutations shift the reading frame of the gene). In some embodiments, the mutant and/or variant of the present disclosure carries at least one mutation that leads to “substitution”, e.g., replacement of at least one amino acid residue with another residue, specifically, replacement of amino acid in position 312 of the ADAMTS-13 protein, and/or of any adjacent amino acid residue/s as discussed herein after, with any other amino acid residue.

Specifically, the invention relates to ADAMTS-13 mutant having a mutation in the amino acid residue Arg312 or in at least one adjacent amino acid residue. As used herein, term “adjacent amino acid residue” refers to an amino acid being at a position of about 1 to 5 or more residues upstream or alternatively, downstream to residue 312 (also referred to herein as minus or plus, respectively). Specifically, at least one, at least two, at least three, at least four, at least five or more residues upstream of residue 312, specifically, residues 307, 308, 309, 310, 311. Or alternatively, at least one, at least two, at least three, at least four, at least five or more residues downstream of residue 312, specifically, residues 313, 314, 315, 316, 317. In more specific embodiments, the mutants of the invention may carry a substitution of residue 312, or of an amino acid residue located two residues plus/minus to residue 312. For example, in some embodiments, amino acid residue adjacent to Arg312 corresponds to amino acid residue 310, 311, 313 or 314. In some specific embodiments, an adjacent amino acid residue to Arginine 312 may be Valine 313. Thus, in some embodiments, the mutant/s and or variant/s disclosed herein may carry a mutation that leads to substitution and/or replacement of amino acid residue at position 312. In yet some further embodiments, the mutant of the present disclosure may carry at least one mutation that substitutes at least one residue adjacent to residue 312, for example, a mutation that result in substitution of at least one of residues 307, 308, 309, 310, 311, 313, 314, 315, 316, 317, with another amino acid residue. Still further, in some embodiments, the mutant and/or variant/s disclosed herein may carry at least one mutation replacing amino acid residues at position 312 and in addition, a substitution of at least one of the following residues 307, 308, 309, 310, 311, 313, 314, 315, 316, 317, of ADAMTS-13 protein, specifically as denoted by SEQ ID NO: 2. In yet some specific embodiments, the mutant of the present invention may carry at least one mutation that substitutes residues 312 and 313 of ADAMTS-13.

As indicated above, the ADAMTS-13 mutants of the invention comprise replacement of residue 312 and/or any adjacent residue with any charged amino acid residue. A charged amino acid residue may be residues that are either negative (i.e. de-protonated) at physiological pH, for example, aspartic acid (Asp, D) and glutamic acid (Glu, E), or positive (i.e. protonated) at physiological pH, for example, the lysine (Ly, K), arginine (Arg, R) and histidine (His, H).

In some embodiments, the charged amino acid residue of the replacing specific amino acid residues in the ADAMTS-13 mutant or any truncated variant thereof may be any one of lysine (K), aspartic acid (D), glutamic acid (E) or histidine (H).

In some specific embodiments, the charged amino acid residue of the ADAMTS-13 mutant or any truncated variant thereof may be lysine.

In some embodiments, the ADAMTS-13 mutant may carry a mutation substituting the Arginine in position 312 with any amino acid residue, with the proviso that said amino acid reside is not Alanine.

In yet some further specific embodiments, the ADAMTS-13 mutant of the present disclosure may carry a mutation substituting the Arginine in position 312 to lysine. Such mutant is designated R312K. In some embodiments, the mutant may comprise the amino acid sequence as denoted by SEQ ID NO:11 or any variants, homologues or derivatives thereof.

In some embodiments, the present disclosure provides mutants or variants of ADAMTS-13, and/or any variants of the disclosed mutants. Variants of the disclosed mutants may encompass any truncated, extended, modified variants of the disclosed mutants. Further variants encompassed by the present disclosure are defined herein below.

Thus, in some embodiments, the further disclosure encompasses a truncated variant of the disclosed mutants. A truncated variant of a given protein as used herein refers to any variant comprising an amino acid sequence that was shortened, trimmed, cut or reduced in at least one amino acid residue of the N-terminal end and/or the C-terminal end of the protein. More specifically, the variants encompassed by the present disclosure are truncated variants that are shortened in at least about 1 to about 800 or more residues, either in the C′- and/or N′-termini thereof, specifically, 1, 10, 20, 30, 40, 50, 60, 70, 80, 90, 100, 150, 200, 250, 300, 350, 400, 450, 500, 550, 600, 650, 700, 750, 800 amino acid residues (aa's) or more. In some embodiments, the ADAMTS-13 truncated variant in accordance with the present disclosure is truncated in the N and the C′ termini thereof. Still further, the truncated variant of the mutants disclosed herein are truncated in the C-terminal end of the ADAMTS-13 molecule. In yet some further embodiments, the truncated mutants disclosed herein are truncated in at least one of the following domains: at least one of the thrombospondin type 1 (TSP) repeats 2-8, and the two 2 C-terminal CUB domains (CUB). Still further, in some embodiments, the truncated variant of the present disclosure has a deletion or truncation of the C′ terminal CUB domain. In yet some further embodiments, the two C-terminal CUB domains are truncated in the mutant. Still further, in some embodiments, the truncated mutant is truncated in the two C-terminal CUB domains and TSP repeat 8. In some further embodiments, the truncated mutant is truncated in the two C-terminal CUB domains and TSP repeats 8 and 7. In some further embodiments, the truncated mutant is truncated in the two C-terminal CUB domains and TSP repeats 8, 7 and 6. In some further embodiments, the truncated mutant is truncated in the two C-terminal CUB domains and TSP repeats 8, 7, 6 and 5. In some further embodiments, the truncated mutant is truncated in the two C-terminal CUB domains and TSP repeats 8, 7, 6, 5 and 4. In some further embodiments, the truncated mutant is truncated in the two C-terminal CUB domains and TSP repeats 8, 7, 6, 5, 4 and 3. In some further embodiments, the truncated mutant is truncated in the two C-terminal CUB domains and TSP repeats 8, 7, 6, 5, 4, 3 and 2. In some embodiments, the truncated mutant of the present disclosure comprises the following domains of ADAMTS-13, the metalloprotease (MP), Disintegrin-like domain (Dis), thrombospondin type 1 (TSP) repeat (1) and cysteine-rich and spacer domain (Cys).

In yet some further particular and non-limiting embodiments, a truncated variant of the mutant of the invention may comprise the amino acid sequence as denoted by SEQ ID NO:5, or any variants or derivatives thereof. Accordingly, in some further embodiments, the truncated ADAMTS-13 mutant of the invention may be encoded by a nucleic acid sequence as denoted by SEQ ID NO:18, or any variants or derivatives thereof. Still further, as will be indicated in more detail herein after, truncated variants disclosed by the present disclosure include, but are not limited to any variants comprising the amino acid sequence as denoted by any one of; SEQ ID NO: 7 (truncated WT), SEQ ID NO: 15 (truncated ADAMTS-13^{R312A}), SEQ ID NO: 16 (truncated ADAMTS-13^{V313A}), SEQ ID NO: 17 (truncated V313D), any variants, derivatives thereof, and any combinations thereof.

Still further, it should be understood that the present disclosure encompasses any ADAMTS-13 mutant and/or variant, specifically any truncated variants that comprise at least the MP catalytic domain of the molecule, and therefore retain the ability of cleaving vWF. Still further, the mutants of the present disclosure or any variants thereof retain resistance and/or reduced sensitivity to cleavage and/or inactivation by at least one proteolytic protein, specifically, by tPA.

As indicated above, the mutants or variants of the present disclosure may comprise at least one mutation in residue 312, and/or in any adjacent residue. Thus, in some further embodiments, the adjacent amino acid residue of the ADAMTS-13 mutant of the invention or any truncated variant thereof, may be valine 313. In such embodiments, the mutant may carry a mutation substituting valine 313 with aspartic acid. In some embodiment, the mutant may be designated V313D. In yet some further embodiments, such mutant may comprise the amino acid sequence as denoted by SEQ ID NO: 14 or any derivative or variants thereof. In yet some further embodiments, a truncated version of such mutant may comprise the amino acid sequence as denoted by SEQ ID NO: 17, or any derivative or variants thereof. Such truncated mutant may be encoded in some embodiments by the nucleic acid sequence as denoted by SEQ ID NO: 21. In yet some specific and non-limiting embodiments, the mutants disclosed herein may comprise substitution of resides 312 and 313. Thus, in some embodiments, such double mutant may comprise R312K and V313D. In some embodiments, such double mutants may comprise the amino acid sequence as denoted by SEQ ID NO: 23 and the truncated form thereof as denoted by SEQ ID NO; 24.

Still further, ADAMTS-13 mutants encompassed by the invention include the R312A mutant (replacing Arg 312 with alanine), such mutant may comprise the amino acid sequence as denoted by SEQ ID NO: 12 or any derivative or variants thereof, and the truncated version thereof may comprise the amino acid sequence as denoted by SEQ ID NO: 15 or any derivative or variants thereof. It yet some further embodiments, the truncated version of the R312A mutant may be encoded by a nucleic acid sequence comprising SEQ ID NO: 19.

In some further embodiments, the invention further encompasses ADAMTS-13 mutant that carry a replacement of valine 313 with alanine V313A. Such mutant may comprise the amino acid sequence as denoted by SEQ ID NO: 13 or any derivative or variants thereof, and the truncated version thereof may comprise the amino acid sequence as denoted by SEQ ID NO: 16 or any derivative or variants thereof. It yet some further embodiments, the truncated version of the R312A mutant may be encoded by a nucleic acid sequence comprising SEQ ID NO: 22.

In yet some further embodiments, the invention provides ADAMTS-13 variants that are truncated variant comprising the amino acid sequence as denoted by SEQ ID NO: 7 or any derivative or variants thereof, encoded by the nucleic acid sequence as denoted by SEQ ID NO: 6.

As indicated above, the invention provides ADAMTS-13 mutants and variants thereof, specifically, as denoted by SEQ ID NO: 5, 11, 12, 13, 14, 15, 16, 17, 23 and 24, or any variants or derivatives thereof. As indicated above, all mutants and variants disclosed herein, are functional mutant and/or variant in the sense of they all retain the ability of cleaving vWF, and/or they all retain resistance and/or reduced sensitivity to cleavage and/or inactivation by tPA. Still further, in some embodiments, the mutants of the present disclosure retain ability of dissolving blood clots.

It should be appreciated that the invention encompasses any variant or derivative of the ADAMTS-13 mutant polypeptides of the invention and any polypeptides that are substantially identical or homologue to the polypeptides encoded by the nucleic acid sequence of the invention. The term “derivative” or “variant” is used to define amino acid sequences (polypeptide), with any insertions, deletions, substitutions and modifications to the amino acid sequences (polypeptide) that do not alter the activity of the original polypeptides. Specifically, ability of cleaving vWF, and/or resistance and/or reduced sensitivity to cleavage and/or inactivation by tPA. By the term “derivative” it is also referred to homologues, variants and analogues thereof. Proteins orthologs or homologues having a sequence homology or identity to the proteins of interest in accordance with the invention, specifically, all ADAMTS-13 mutants and/or variants described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the proteins that comprise the amino acid sequence as denoted by any one of SEQ ID NO: 5, 11, 12, 13, 14, 15, 16, 17, 23 and 24. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to the entire sequence of the mutants and/or variants of the invention, specifically, any of the mutants that comprise the amino acid sequence as denoted by SEQ ID NO: 5, 11, 12, 13, 14, 15, 16, 17, 23, 24 and any derivatives, homologues and variants thereof.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Thus, in some embodiments, the present disclosure encompasses the specified polypeptides, or any derivatives thereof, specifically a derivative that comprise at least 1, 2, 3, 4, 5, 6, 7, 8, 9, 10 or more conservative substitutions to the amino acid sequences as denoted by any one of SEQ ID NO: 5, 11, 12, 13, 14, 15, 16, 17, 23 24. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the entire protein of interest, such as the various polypeptides of the invention.

As shown in Examples 2 and 3, the inventors have first demonstrated that tPA cleaves the ADAMTS-13 protein and inhibits its activity. The exact cleavage site was determined to be at R³¹²-V³¹³. Several mutants perturbing the R312V313 bond in ADAMTS-13 were created, some of them exhibiting resistance to cleavage to tPA, as demonstrated in Example 4. Therefore, in some embodiments, as discussed herein above, the ADAMTS-13 mutant of the invention or any truncated variant thereof display resistance, and/or reduced sensitivity to cleavage by at least one serine protease.

In certain embodiment, the serine protease may be at least one of tissue plasminogen activator (tPA), urokinase plasminogen activator (uPA), plasmin, thrombin or granulocyte elastase, or other plasminogen activators like streptokinase or any functional fragments or variants thereof.

In some specific embodiments, the ADAMTS-13 mutant of the invention or any truncated variant thereof may display resistance and/or reduced sensitivity to cleavage and/or inactivation by at least one tissue plasminogen activator (tPA), or any mutant or variant thereof.

It should be appreciated that the term tPA used herein for the tissue plasminogen activator (also known as PLAT; enzyme entry EC 3.4.21.68), relates to a secreted serine protease that converts and activates the proenzyme plasminogen to a potent fibrinolytic enzyme plasmin. tPA is synthesized in vascular endothelial cells as a single polypeptide chain that undergoes proteolytic cleavage by plasmin or trypsin at a centrally located arginine-isoleucine bond, resulting in a 2-chain disulfide-linked form composed of the N-terminally derived heavy chain and the C-terminal light chain. The tPA gene (DNA acc. NT_167187.1 mapped to chr. 8p11.21) contains 14 exons encoding the heavy chain domain including two kringle regions (K1 and K2) and regions homologous to growth factors and the light chain domain comprising the serine protease catalytic site. Alternative splicing of the tPA gene results in multiple transcript variants encoding different isoforms taking part in multiple biological processes, apart from fibrinolysis, such as cell migration and tissue remodeling. Increased tPA activity causes hyperfibrinolysis manifested as excessive bleeding; decreased tPA activity leads to hypofibrinolysis which can result in thrombosis or embolism. tPA linked phenotypes include familial hyperfibrinolysis (due to increased tPA release) and familial thrombophilia (due to decreased tPA release (OMIM num. 612348). It should be noted that in some embodiments, tPA, as used herein refers to the human tPA that comprise the amino acid sequence encoded by the nucleic acid sequence comprising the sequence as denoted by SEQ ID NO: 3, or any variants or derivatives thereof. In some further embodiments, the human tPA may comprise an amino acid sequence encoded by a nucleic acid sequence comprising a sequence having at least about 70%, 75%, 80%, 85%, 90%, 91%, 92%, 93%, 94%, 95%, 96%, 97%, 98%, 99% or 100% homology with the sequence as denoted by SEQ ID NO: 3. In yet some further embodiments, such human tPA molecule may comprise the amino acid sequence as denoted by SEQ ID NO: 4. In yet some other embodiments, the human tPA may comprise an amino acid sequence having at least about 70%, 75%, 80%, 85%, 90%, 91%, 92%, 93%, 94%, 95%, 96%, 97%, 98%, 99% or 100% homology with the sequence as denoted by SEQ ID NO: 4, and any variants, homologues and derivatives thereof.

As indicated herein, the disclosed mutants and variants display resistance to cleavage, specifically, proteolytic cleavage (e.g., proteolysis) by at least one serine protease such as tPA. Proteolysis, as used herein is the breakdown of proteins into smaller polypeptides or amino acids. In some embodiments, resistance and/or reduced sensitivity to proteolytic cleavage as used herein is meant that the ADAMTS-13 mutant disclosed herein is less vulnerable, and/or displays reduced vulnerability and/or sensitivity and/or increased resistance to proteolytic cleavage and/or inactivation by at least about 5% to 100% or more. In some embodiments, reduced sensitivity and/or vulnerability is meant that about 5% to 100% of the mutated ADAMTS-13 molecule is not cleaved and/or inactivated by tPA. More specifically, in some embodiments, the ADAMTS-13 mutant of the present disclosure display reduced sensitivity to cleavage and/or inactivation by tPA, specifically, a reduced sensitivity of at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more as compared to the sensitivity of the wild type ADAMTS-13. Still further, the mutant of the present disclosure display resistance or increased resistance to cleavage, degradation and/or inactivation by tPA, specifically, an increase of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more resistance to cleavage, or alternatively, reduced sensitivity as compared to the wild type ADAMTS-13. Inactivation, as referred to herein, is meant any reduction of about 5% to about 100%, in the disclosed activity of ADAMTS-13, e.g., proteolysis of any substrate, specifically, the vWF, speed and extent of dissolving blood clots and restoring blood flow, as compared to the wild type ADMTS-13. Still further, in some embodiments, about 5% to 100% of the mutated ADAMTS-13 of the present disclosure in a given composition or any biological fluid (e.g., blood, plasma, either in a subject or in vitro), is not cleaved and/or inactivated by tPA present in the same biological fluid.

Still further, in some embodiments, the ADAMTS-13 mutant and/or variant provided herein, or any truncated variant thereof, display an increased or enhanced activity, as compared to the WT ADAMTS-13. In yet some other embodiments, the ADAMTS-13 mutant of the invention may exhibit enhanced enzymatic activity. In some specific embodiment, the enzymatic activity of the ADAMTS-13 mutant of the invention may refer to cleavage of uWF. According to some embodiments, wherein indicated “increased” or “enhanced” activity, it is meant that such increase or enhancement may be an increase or elevation of between about 10% to 100% of the ADAMTS-13 mutant activity in comparison with the Wild type ADAMTS-13 protein. The terms “increased”, “augmented” and “enhanced” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the activity as compared to a suitable control, e.g., the Wild type ADAMTS-13 protein. It should be further noted that increase or elevation may be also an increase of about 2 to 10⁶ folds or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. Therefore, the term increase refers to an increase of about 2, 3, 4, 5, 6, 7, 8, 9, 10, 20, 30, 40, 50, 60, 70, 80, 90, 100, 200, 300, 400, 500, 600, 700, 800, 900, 1000 folds or more.

As noted above, the ADAMTS-13 mutants of the invention display enhanced cleavage of vWF. von Willebrand factor (VWF) is a blood glycoprotein involved in hemostasis. VWF is a large multimeric glycoprotein present in blood plasma and produced constitutively as ultra-large VWF in endothelium (in the Weibel-Palade bodies), megakaryocytes (α-granules of platelets), and subendothelial connective tissue. The basic VWF monomer is a 2050-amino acid protein. Every monomer contains a number of specific domains with a specific function; elements of note include:
the D′/D3 domain, which binds to factor VIII (von Willebrand factor type D domain);
the A1 domain, which binds to: platelet GPIb-receptor, heparin and possibly collagen;
the A2 domain, which must partially unfold to expose the buried cleavage site for the specific ADAMTS-13 protease that inactivates VWF by making much smaller multimers (the partial unfolding is affected by shear flow in the blood, by calcium binding, and by the lump of a sequence-adjacent “vicinal disulfide” at the A2-domain C-terminus);
the A3 domain, which binds to collagen (von Willebrand factor type A domain);
the C4 domain, in which the RGD motif binds to platelet integrin αIIbβ3 when this is activated (von Willebrand factor type C domain);
the other C domains, which may interact in ER dimers: the larger protein show six beads of (C and C-like) domains under cryo-EM;
the “cystine knot” domain (at the C-terminal end of the protein), which VWF shares with platelet-derived growth factor (PDGF), transforming growth factor-β (TGFβ) and β-human chorionic gonadotropin (PHCG, of pregnancy test fame), (von Willebrand factor type C domain);

Monomers are subsequently N-glycosylated, arranged into dimers in the endoplasmic reticulum and into multimers in the Golgi apparatus by crosslinking of cysteine residues via disulfide bonds. With respect to the glycosylation, VWF is one of only a few proteins that carry ABO blood group system antigens. vWFs coming out of the Golgi are packaged into storage organelles, Weibel-Palade bodies (WPBs) in endothelial cells and α-granules in platelets.

Multimers of VWF can be extremely large, >20,000 kDa, and consist of over 80 subunits of 250 kDa each. Only the large multimers are functional.

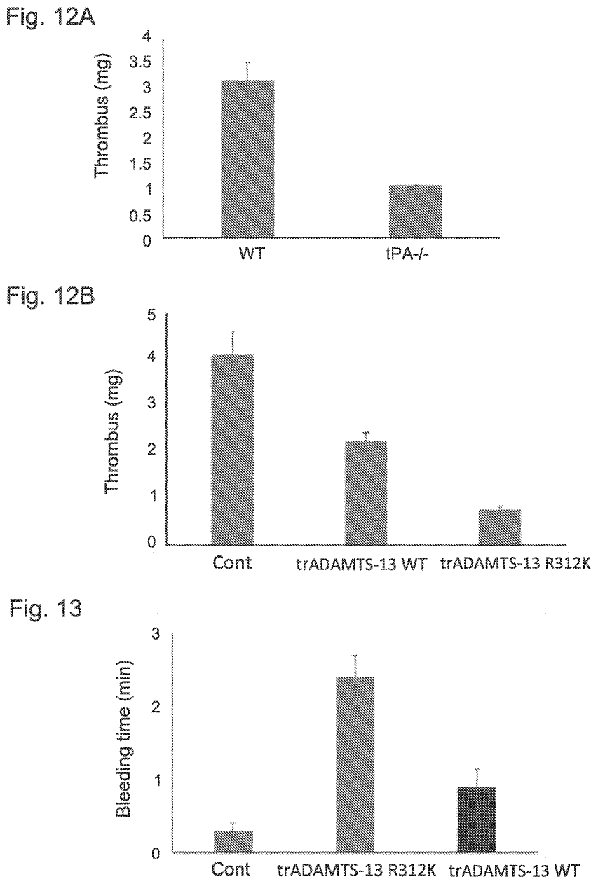

In yet some further alternative and/or additional embodiments, increased activity when referred to the ADAMTS-13 mutant/s and/or variant/s disclosed herein, may relate to the ability of the mutant/s and/or variant/s of the present disclosure to reduce size and/or volume and/or weight of at least one blood clot, in about 5%, 10%, 15%, 20%, 25%, 30%, 35%, 40%, 45%, 50%, 55%, 60%, 65%, 70%, 75%, 80%, 85%, 90%, 95%, 100%, or more, as compared with the ability of the wild type ADAMTS-13 to reduce size and/or volume and/or weight of at least one blood clot. Enhanced activity (two folds or more) of the mutant of the present invention to dissolve blood clot is shown by FIG. 12. Still further, in some embodiments enhanced activity of the disclosed ADAMTS-13 mutant/s and/or variant/s may refer to reduced time of dissolving blood clots, and/or time of restoring blood flow, as also clearly exemplified by FIG. 10 and Example 6. More specifically, in some embodiments, “time of restoring blood flow” as used herein, is meant the time (e.g., minutes) required for recovery from a reduction to about 25% or less (and even cessation) of the blood flow (e.g., the blood volume passing over time through a blood vessel). In some embodiments, the reduction in blood flow is occurred for example by occlusive thrombus or blood clot. Still further, the time required to restore the blood flow to about 30%, 35%, 40%, 45%, 50%, 55%, 60%, 65%, 70%, 75%, 80%, 85%, 90%, 95% and 100% of the blood flow before occlusion or blockage occurred.

In some embodiments, the ADAMTS-13 mutants disclosed herein and any compositions thereof significantly reduce the time required for restoring the blood flow and/or dissolving the blood clot, when administered to a subject.

A thrombus, colloquially called a blood clot, as used herein, is the final product of blood coagulation. The two major components to a thrombus are aggregated platelets and red blood cells that form a plug, and a mesh of cross-linked fibrin protein. Thrombi are classified into two major groups depending on their location and relative amount of platelets and red blood cells. More specifically, arterial or white thrombi (characterized by predominance of platelets), and venous or red thrombi (characterized by predominance of red blood cells). An occlusive thrombus is a blockage that completely seals off a blood vessel, where a non-occlusive thrombus only partially blocks off a blood vessel (e.g., vein or artery).

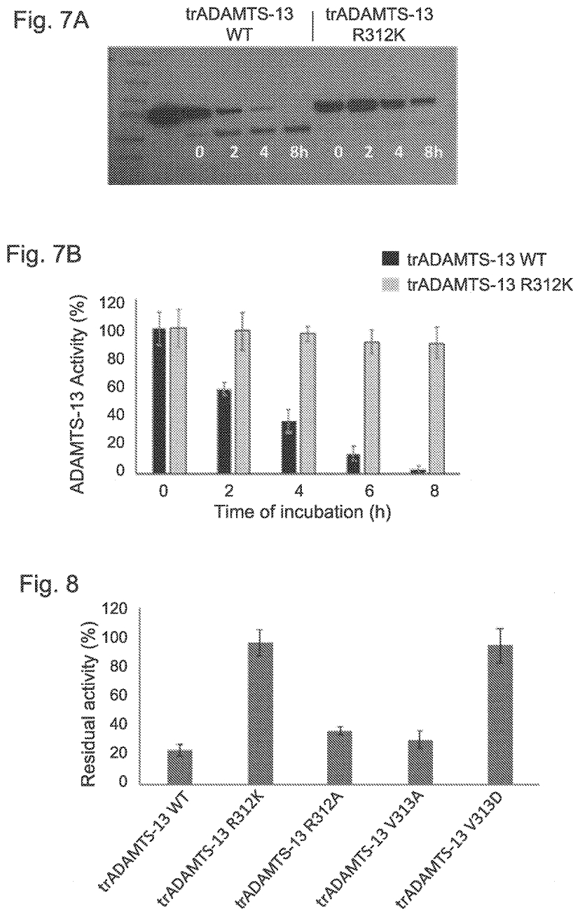

As shown by FIG. 7 and Example 4, the mutant/s and/or variant/s disclosed herein, specifically, the ADAMTS-13^{R312K} mutant, when compared to the WT ADAMTS-13, displayed extended duration of proteolytic activity (e.g., 8 hr to 24 hr vs 2 hr to 6 hr) as reflected by proteolysis of vWF. These results indicate increased stability and activity over time, as defined by a prolonged half-life of the disclosed mutants and are clearly valuable for therapeutic applications. Indeed, as disclosed in Example 4, the half-life of the disclosed mutant trADAMTS-13^{R312K}, is about 344.6+/−131.1 min, whereas the half-life of the wild-type molecule is much lower (132.8+/−41.3 min). In some embodiment, the half-life of the mutant trADAMTS-13^{R312K} is between about 150 to about 750 minutes or more, specifically, about 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750 minutes or more.

Thus, in yet some further embodiments, the ADAMTS-13 mutant and/or variant disclosed herein or any truncated variant thereof, display a prolonged half-life relative to ADAMTS-13 wild type. In some embodiments, the prolonged half-life of the ADAMTS-13 mutant disclosed herein extends the duration of the ADAMTS activity, as reflected by proteolysis of vWF, up to 24 hr or more, specifically, up to 1, 2, 3, 4, 5, 6, 7, 8, 9, 10, 11, 12, 13, 14, 15, 16, 17, 18, 19, 20, 21, 22, 23, 24 hrs or more. The mutants of the invention therefore display in some embodiments an extended activity window. A biological half-life (also known as elimination half-life, pharmacologic half-life), also denoted by the abbreviation of a biological substance such as the ADAMTS-13 mutant of the present disclosure, is the time it takes to reduce the maximum concentration (Cₘₐₓ) to half of its maximum concentration in the blood plasma in about 1% to about 100% or more. More specifically, the ADAMTS-13 mutants of the present disclosure display a half-life that is prolonged in about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more. It is to be understood that, where provided, percentage values such as, for example, 10%, 50%, 120%, 500%, etc., are interchangeable with “fold change” values, i.e., 0.1, 0.5, 1.2, 5, etc., respectively. Therefore, the term prolonged refers to half-life increased in about 2, 3, 4, 5, 6, 7, 8, 9, 10, 20, 30, 40, 50, 60, 70, 80, 90, 100, 200, 300, 400, 500, 600, 700, 800, 900, 1000 folds or more.

Still Further, additional beneficial effects of the ADAMTS-13 mutants of the invention relate to resistance to anti ADAMTS-13 autoantibodies found for example in plasma of Thrombotic Thrombocytopenic Purpura (TTP) patients, as shown in Example 5. Therefore, in some embodiments, the ADAMTS-13 mutant of the invention may exhibit resistance to anti ADAMTS-13 autoantibodies.

It should be understood that the various properties of the disclosed ADAMTS-13 mutants as discussed herein are applicable for any of the ADAMTS-13 mutant/s and/or variant/s disclosed in any of the aspects of the present disclosure.

The invention provides mutants of ADAMTS 13 protein. The terms “protein” or “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

As indicated herein, the present disclosure provides various ADAMTS-13 mutants and/or variant. It should be appreciated however, that the invention further encompasses any nucleic acid sequence encoding the mutants and/or variant disclosed herein. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs. Preparation of nucleic acids is well known in the art. Moreover, the present disclosure further provides any vector, expression vector or any construct comprising at least one nucleic acid molecule encoding the claimed mutants or variants. Vectors, as used herein, are nucleic acid molecules of particular sequence that can be introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g., plasmids, cosmids, minicircles, phage, viruses, (as detailed below) useful for transferring nucleic acids into target cells may be applicable in the present invention. The vectors comprising the nucleic acid(s) may be maintained episomally, e.g., as plasmids, minicircle DNAs, viruses such cytomegalovirus, adenovirus, or they may be integrated into the target cell genome, through homologous recombination or random integration.

Still further, in some embodiments, the preset disclosure provides any host cell expressing any nucleic acid sequence that encodes the ADAMTS-13 mutants and variant/s disclosed herein.

The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell, as well as any population of cells comprising the host cell/s of the invention. Because certain modifications may occur in succeeding generations due to either mutation or environmental influences, such progeny may not, in fact, be identical to the parent cell, but are still included within the scope of the term “host cell”. The term “host cells” as used herein refers to any cell known to a skilled person wherein the ADAMTS-13 variant and/or mutated molecule or any functional fragments or peptides thereof or any nucleic acid molecule according to the invention may be introduced. For example, a host cell may be eukaryotic or prokaryotic cell of a unicellular or multi-cellular organism. More specifically, a host cell may include, but is not limited to a yeast, fungi, an insect cell, an invertebrate cell, vertebrate cell, mammalian cell and the like.

It should be understood that in some embodiments, the ADAMTS-13 mutants of the present disclosure may be produced either recombinantly, for example by expressing the encoding nucleic acid sequence or any vector thereof, in any appropriate host cell, for example, any of the host cells disclosed herein.

In yet some other aspect, the invention relates to a composition comprising an effective amount of at least one ADAMTS-13 mutant, or any truncated variant thereof. In some embodiments, the mutant/s and/or variants of the composition disclosed herein may display resistance and/or reduced sensitivity to cleavage and/or inactivation by at least one seine protease, for example, tPA.

In some embodiments, the mutant may carry a mutation and at least one of the mutation/s may substitute the Arg312 residue and/or any amino acid residue adjacent to the Arg312 of the wild type ADAMTS-13 with a charged amino acid residue. It should be noted that the wild type ADAMTS-13 comprises the amino acid sequence as denoted by SEQ ID NO: 2, and therefore the position 312, refers to SEQ ID NO: 2. In some embodiments, the composition may optionally further comprise at least one pharmaceutically acceptable carrier, diluent, excipient and/or additive.

In some other embodiments, the compositions of the invention or any combined compositions may comprise any of the ADAMTS-13 mutants disclosed by the invention, as specifically described above.

In some further embodiments, the composition of the invention may further comprise a therapeutically effective amount of at least one serine protease. In some alternative embodiments, the serine protease may be at least one of tPA, uPA, plasmin, thrombin or granulocyte elastase, or other plasminogen activators like streptokinase or any functional fragments or variants thereof.

In some specific embodiments, the composition of the invention may further comprise a therapeutically effective amount of at least one tissue plasminogen activator (tPA), or any mutant or variant thereof.

“Pharmaceutically or therapeutically acceptable carrier” refers to a carrier medium which does not interfere with the effectiveness of the biological activity of the active ingredients. As mentioned herein, the compositions provided by the invention optionally further comprise at least one pharmaceutically acceptable excipient or carrier. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

As used herein “pharmaceutically acceptable carrier/diluents/excipient” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

In various embodiments, the final solution of any of the compositions of the invention may be adjusted with a pharmacologically acceptable acid, base or buffer. In some embodiments, the compositions of the invention may be suitable for systemic administration. The pharmaceutical composition of the invention can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (MDI's), or dry powder dispersion devices.

In other embodiments the pharmaceutical composition may be adapted for topical administration. By “topical administration” it is meant that the pharmaceutical composition and the carrier may be adapted to any mode of topical administration including: epicutaneous, transdermal, oral, bronchoalveolar lavage, ophtalmic administration, enema, nasal administration, administration to the ear, administration by inhalation.

Regardless of the route of administration selected, the compositions of the present invention, which may be used in a suitable hydrated form, and/or the pharmaceutical compositions of the present invention, are formulated into pharmaceutically-acceptable dosage forms by conventional methods known to those of skill in the art.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, and suitable mixtures thereof. The proper fluidity can be maintained, for example, by the use of a coating, such as lecithin, by the maintenance of the required particle size in the case of dispersion and by the use of surfactants.

The fact that the ADAMTS-13 mutants of the invention display resistance to cleavage and/or inactivity by tPA, is of particular interest for combined composition suitable for conditions that necessitate the cleavage by both enzymes. Indeed, as shown, also in Example 6, the ADAMTS-13 mutants developed by the inventors exhibit a synergistic activity with tPA on cleavage vWF-rich clots. As indicated herein, the ADAMTS-13 mutants of the invention, as well as in the compositions, combined compositions and kits of the invention, and used by the methods described herein after, may act in synergy with tPA. Therefore, in some further embodiments, the ADAMTS-13 mutant of the invention may exhibit synergistic activity with tPA.

Synergy, as used herein refer to the interaction or cooperation of two or more substances, compounds, or any other agents, specifically, at least one ADAMTS-13 mutants of the invention and tPA or any functional fragments or variants thereof, to produce a combined effect greater than the sum of their separate effects.

Thus, in another aspect, the invention relates to a combined composition comprising a combination of at least one ADAMTS-13 mutant or any truncated variant thereof, and at least one tPA or any functional fragments or variants thereof. In some embodiments, the mutant may carry at least one mutation. In some embodiments, the mutant/s and/or variants of the combined composition display resistance and/or reduced sensitivity to cleavage and/or inactivation by at least one serine protease, specifically, tPA.

In yet some further embodiments, the combined composition of the present disclosure comprises at least one ADAMTS-13 mutant and/or variant that carry at least one mutation that substitute the Arg312 residue and/or any amino acid residue adjacent to said Arg312 of the wild type ADAMTS-13 with a charged amino acid residue. It should be noted that the wild type ADAMTS-13 may comprise the amino acid sequence as denoted by SEQ ID NO: 2, or any variants or derivatives thereof. Still further, in some embodiments, the composition may optionally further comprise at least one pharmaceutically acceptable carrier, diluent, excipient and/or additive.

In some embodiments, the mutant comprised in the combined composition of the invention may be any of the ADAMTS-13 mutants of the invention, specifically, any of the mutants defined above, or any combinations thereof. It should be understood that the tPA comprised within the combined composition disclosed herein is in some embodiments, the tPA as disclosed herein above in connection with other aspects of the invention.

In some further embodiments, the combined composition of the invention may further or alternatively, comprise a therapeutically effective amount of at least one additional or alternative serine protease. In some alternative embodiments, the serine protease may be at least one of uPA, plasmin, thrombin or granulocyte elastase, or other plasminogen activators like streptokinase or any functional fragments or variants thereof.

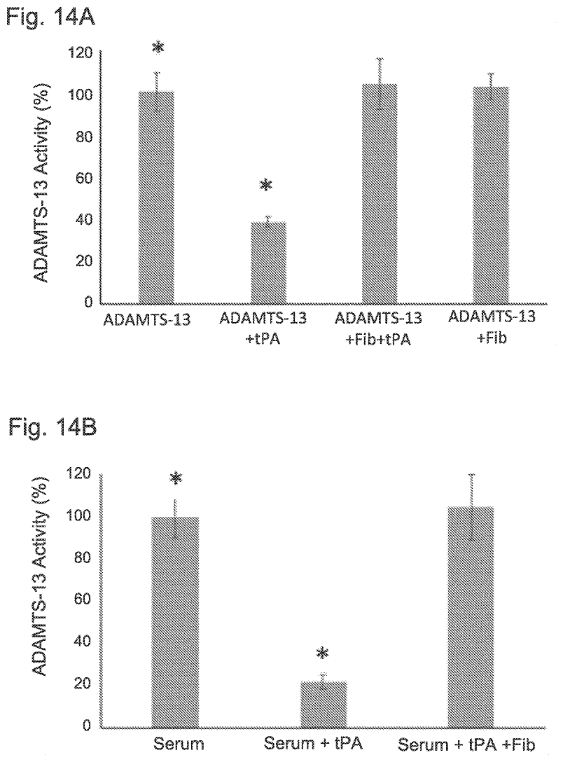

Still further, as shown by FIG. 14 and Example 8, the presence of fibrin led to reduced cleavage and/or inactivation of ADAMTS-13 by tPA and therefore enhanced the activity of ADAMTS-13 on vWF. Thus, in some specific embodiments, the combined composition may further comprise in addition to at least one ADAMTS-13 mutant/s and/or variant/s, and tPA, also fibrin.

Still further, in yet some further embodiments, the present disclosure further encompasses compositions comprising wild type ADAMTS-13, tPA and fibrin, and further encompasses any use of this combined composition, as discussed in the present disclosure.

The anti-thrombotic activity of the ADAMTS-13 mutants was also demonstrated in vivo in mice, as shown in Example 7. Mice injected with the ADAMTS-13R312K mutant retained greater capacity to proteolyze vWF and an increased effect on bleeding was observed as well, indicating that the mutants of the invention may be used as potent anti-coagulation agents.

Thus, in yet some other embodiments, the mutant/s and/or variant/s of the present disclosure, as well as any composition or combined composition thereof, may be particularly suitable for use in a method for the treatment, amelioration, inhibition or prophylaxis of a disease, disorder, or condition associated with coagulation in a subject in need thereof. Coagulation, also known as clotting, is the process by which blood changes from a liquid to a gel, forming a blood clot. It potentially results in hemostasis, the cessation of blood loss from a damaged vessel, followed by repair. The mechanism of coagulation involves activation, adhesion and aggregation of platelets, as well as deposition and maturation of fibrin. Coagulation begins almost instantly after an injury to the blood vessel has damaged the endothelium lining the blood vessel. Exposure of blood to the subendothelial space initiates two processes: changes in platelets, and the exposure of subendothelial tissue factor to plasma Factor VII, which ultimately leads to cross-linked fibrin formation. Platelets immediately form a plug at the site of injury; this is called primary hemostasis. Secondary hemostasis occurs simultaneously: additional coagulation (clotting) factors beyond Factor VII (listed below) respond in a cascade to form fibrin strands, which strengthen the platelet plug.

Disorders of coagulation are disease states which can result in bleeding—hemorrhage or bruising—or obstructive clotting—thrombosis. As used herein, the term “disease, disorder, or condition associated with coagulation” refers to any condition and/or disorder that relates directly and/or indirectly to obstructive clotting, i.e., excessive unregulated clothing and coagulation process, or alternatively or additionally, to any condition caused by a blood clot located in a blood vessel affecting particular organ, that may lead to ischemic condition. This term will be further defined herein after in connection with additional aspects of the present disclosure.

As discussed above, the in vivo effect of the mutants or variants of the present disclosure on resolving blood clots is applicable in treating any condition associated directly or indirectly with coagulation.

Thus, in a further aspect, the invention relates to methods for the treatment, amelioration, inhibition or prophylaxis of a disease, disorder, or condition associated with coagulation in a subject in need thereof. In some embodiments, the method may comprise the step of administering to the subject a therapeutically effective amount of at least one ADAMTS-13 mutant/s and/or variant (e.g., any truncated variant thereof), or any composition or combined composition comprising the mutant/s and/or variant/s of the invention. In certain embodiments, the mutants of the invention may carry at least one mutation. In certain embodiments, the mutant/s and/or variant/s used by the methods disclosed herein may display resistance and/or reduced sensitivity to cleavage and/or inactivation by at least one serine protease, for example, tPA. In yet some further specific embodiments, the mutant/s used by the disclosed method may carry at least one mutation that substitute the Arg312 residue and/or any amino acid residue adjacent to the Arg312 of the wild type ADAMTS-13 with a charged amino acid residue. It should be noted that the wild type ADAMTS-13 may comprise the amino acid sequence as denoted by SEQ ID NO: 2.

In some embodiments, the charged amino acid residue of the mutant/s and/or variant/s suitable for the method of the invention may be any one of lysine, aspartic acid, glutamic acid or histidine.

In some specific embodiments, the charged amino acid residue of the mutant/s and/or variant/s suitable for the method of the invention may be lysine.

In some more specific embodiments, mutant/s and or variant/s suitable for the methods of the invention may carry a mutation substituting the Arginine in position 312 to lysine and may be designated R312K, or ADAMTS-13R³¹²K. In certain embodiment, the mutant may comprise the amino acid sequence as denoted by SEQ ID NO:11, or any variants or derivatives thereof. In yet some further embodiments, such variants or derivatives may be the truncated variant of the mutant. In some specific embodiments, such truncated variant may comprise the amino acid sequence as denoted by SEQ ID NO:5, or any variant or derivatives thereof. It should be understood that ADAMTS-13 mutant/s and/or variant/s suitable for the present disclosure may be any of the mutant/s and/or variant/s disclosed by the present disclosure in connection with other aspects of the invention.

In some other specific embodiments, any mutant/s or variant/s of ADAMTS-13 disclosed herein is suitable for the method of the invention, with the proviso that the mutant and/or variant does not carry a mutation substituting the Arginine in position 312 to Alanine. In some specific embodiments such mutant may comprise the amino acid sequence as denoted by SEQ ID NO: 12, or the truncated version thereof that comprise the amino acid sequence as denoted by SEQ ID NO: 15. Thus, according to some specific embodiments, the methods disclosed herein may use and therefore administer any of the ADAMTS-13 mutant/s and/or variant/s disclosed herein, provided that such mutants are not the mutants of SEQ ID NO: 5 or of SEQ ID NO: 12.

Still further, in some other embodiments, the adjacent amino acid residue of the mutant suitable for the methods of the invention may be valine 313. In some specific embodiments, such mutant/s that are suitable in the present disclosure may carry a mutation substituting valine 313 with aspartic acid. In some embodiments such mutant may comprise the amino acid sequence as denoted by SEQ ID NO: 14, or the truncated version thereof that comprise the amino acid sequence as denoted by SEQ ID NO: 17.

In some further embodiments, the mutant suitable for the method of the invention may be any of the ADAMTS-13 mutants of the invention, specifically, any of the mutants defined above.

As indicated above, in some other embodiments, the ADAMTS-13 mutant/s and/or variant/s suitable for the method of the invention may display resistance and/or decreased sensitivity to cleavage and/or inactivation by at least one serine protease. In certain embodiments, the serine protease may be at least one of tPA, and/or any other serine protease, for example, uPA, plasmin, thrombin or granulocyte elastase or other plasminogen activators like streptokinase or any functional fragments or variants thereof, specifically, tPA.

Thus, in certain embodiments, the methods of the invention may further comprise the step of administering to the subject, a therapeutically effective amount of at least one tPA or any functional fragments or variants thereof or any composition thereof.

In some embodiments, the mutant/s and/or variant/s used by the methods disclosed herein (e.g., any truncated variant) display an increased activity. It should be understood that an increased activity of the disclosed mutants/and/or variant/s as used herein is as defined in connection with previous aspects of the present disclosure (e.g., proteolysis of vWF, resolving blood clots, recovering blood low, and more).

In yet some further embodiments, the ADAMTS-13 mutant/s or any variant/s thereof (e.g., the truncated variant) used by the methods disclosed herein display a prolonged half-life relative to ADAMTS-13 wild type.

In some embodiments, the methods disclosed herein are particularly suitable for treating disease, disorder, or condition that relates and/or is associated directly or indirectly to coagulation. The normal balance between clot formation and breakdown can be changed by the presence of certain genetic or acquired defects leading to abnormal clot formation. It should be therefore understood that the term “coagulation-related disorders” and/or “disorders and/or conditions associated with coagulation” as used herein, encompass any condition associated directly of indirectly with abnormal clot formation and/or maintenance. Reasons for the clot formation and breakdown processes to be unbalanced toward abnormal clot formation include blood vessel injury, venous stasis (lack of movement of the blood in the veins), and clotting disorders. These three factors make up Virchow's triad. An alteration in any one of these three factors can lead to abnormal clotting. All risk factors for DVT or PE fall into one of these three categories. A venous thromboembolic event (VTE) is either a DVT or PE or both in the same patient. Clotting disorders are present in the majority of patients who have a DVT. D-dimer is a by-product of clot breakdown and is elevated in DVT or PE conditions.

There are two types of clotting disorders. The first is a hereditary disorder and the second is an acquired disorder. The hereditary clotting disorders come are divided in two groups: group 1 is characterized with lack of anti-clotting factors in the blood, while group 2 is characterized with an increased amount of pro-clotting factors in the blood.

Group 1 disorders include anti-thrombin deficiency, protein C deficiency, and protein S deficiency. Group 2 disorders include activated protein C resistance (Factor V Leiden mutation), prothrombin G20210A mutation, and elevated levels of Factors VIII, IX, and XI. In general, the Group 1 disorders are less common but more likely to cause abnormal clotting than Group 2 disorders. It should be appreciated that the methods disclosed herein may be applicable for any coagulation disorder, either hereditary or acquired of group 1 and/or group 2, in any stage or degree of any of these disorders.

In some specific and non-limiting embodiments, the therapeutic methods disclosed herein may be applicable for at least one of deep venous thrombosis (DVT), pulmonary emboli (PE), acute ischemic stroke (AIS), acute myocardial function (AMI), thrombotic thrombocytopenic purpura (TTP), disseminated intravascular coagulation (DIC), hemolytic-uremic syndrome (HUS), cerebral infarction or systemic lupus erythematosus (SLE).

In some embodiments, the methods disclosed herein may be applicable for DVT. As used herein, the term “deep venous thrombosis (DVT)” refers to the formation of a blood clot in a deep vein, most commonly the legs. Symptoms may include pain, swelling, redness, or warmth of the affected area. It should be appreciated that about half of cases have no symptoms. Complications may include pulmonary embolism (PE), as a result of detachment of a clot which travels to the lungs, and post-thrombotic syndrome.

Risk factors for such condition include recent surgery, cancer, trauma, lack of movement, obesity, smoking, hormonal birth control, pregnancy and the period following birth, antiphospholipid syndrome, and certain genetic conditions. Genetic factors include deficiencies of antithrombin, protein C, and protein S, and factor V Leidenmutation. The underlying mechanism typically involves some combination of decreased blood flow rate, increased tendency to clot, and injury to the blood vessel wall.

Individuals suspected of having DVT may be assessed using a clinical prediction rule such as the Wells score. A D-dimer test may also be used to assist with excluding the diagnosis or to signal a need for further testing. Diagnosis is most commonly confirmed by ultrasound of the suspected veins. Together, DVT and pulmonary embolism are known as venous thromboembolism (VTE).

Anticoagulation (blood thinners) is the standard treatment. Typical medications include low-molecular-weight heparin, warfarin, or a direct oral anticoagulant.

Still further, in some embodiments, the methods disclosed herein may be applicable for Pulmonary embolism (PE). PE is a blockage of an artery in the lungs by a substance that has moved from elsewhere in the body through the bloodstream (embolism). Symptoms of a PE may include shortness of breath, chest pain particularly upon breathing in, and coughing up blood. Symptoms of a blood clot in the leg may also be present, such as a red, warm, swollen, and painful leg. Signs of a PE include low blood oxygen levels, rapid breathing, rapid heart rate, and sometimes a mild fever. Severe cases can lead to passing out, abnormally low blood pressure, and sudden death.

PE usually results from a blood clot in the leg that travels to the lung. The risk of blood clots is increased by cancer, prolonged bed rest, smoking, stroke, certain genetic conditions, estrogen-based medication, pregnancy, obesity, and after some types of surgery. A small proportion of cases are due to the embolization of air, fat, or amniotic fluid. Diagnosis is based on signs and symptoms in combination with test results. If the risk is low, a blood test known as a D-dimermay rule out the condition. Otherwise, a CT pulmonary angiography, lung ventilation/perfusion scan, or ultrasound of the legs may confirm the diagnosis.

In some embodiments, the methods of the invention may be applicable for AIS. Acute ischemic stroke (AIS) occurs when there is a sudden occlusion of the arterial blood supply to part of the brain and is most commonly manifested by focal neurological deficits.

In an ischemic stroke, blood supply to part of the brain is decreased, leading to dysfunction of the brain tissue in that area. There are several major reasons for stroke, including, thrombosis (obstruction of a blood vessel by a blood clot forming locally), embolism (obstruction due to an embolus from elsewhere in the body), systemic hypoperfusion (general decrease in blood supply, e.g., in shock) and cerebral venous sinus thrombosis.

A stroke without an obvious explanation is termed cryptogenic (of unknown origin), and constitutes 30-40% of all ischemic strokes.

In some embodiments, the methods of the invention may be applicable for acute ischemic stroke. There are various classification systems for acute ischemic stroke. The Oxford Community Stroke Project classification (OCSP, also known as the Bamford or Oxford classification) relies primarily on the initial symptoms; based on the extent of the symptoms, the stroke episode is classified as total anterior circulation infarct (TACI), partial anterior circulation infarct (PACI), lacunar infarct (LACI) or posterior circulation infarct (POCI). These four entities predict the extent of the stroke, the area of the brain that is affected, the underlying cause, and the prognosis. The TOAST (Trial of Org 10172 in Acute Stroke Treatment) classification is based on clinical symptoms as well as results of further investigations; on this basis, a stroke is classified as being due to (1) thrombosis or embolism due to atherosclerosis of a large artery, (2) an embolism originating in the heart, (3) complete blockage of a small blood vessel, (4) other determined cause, (5) undetermined cause (two possible causes, no cause identified, or incomplete investigation). Users of stimulants, such as cocaine and methamphetamine are at a high risk for ischemic strokes.

In some embodiments, the methods of the invention may be applicable for AMI. Acute Myocardial Infarction (AMI) refers to tissue death (infarction) of the heart muscle (myocardium). It is a type of acute coronary syndrome, which describes a sudden or short-term change in symptoms related to blood flow to the heart. Unlike other causes of acute coronary syndromes, such as unstable angina, a myocardial infarction occurs when there is cell death, as measured by a blood test for biomarkers (the cardiac protein troponin or the cardiac enzyme CK-MB). When there is evidence of an MI, it may be classified as an ST elevation myocardial infarction (STEMI) or non-ST elevation myocardial infarction (NSTEMI) based on the results of an ECG.

The phrase “heart attack” is often used non-specifically to refer to a myocardial infarction and to sudden cardiac death. An MI is different from—but can cause—cardiac arrest, where the heart is not contracting at all or so poorly that all vital organs cease to function, thus causing death. It is also distinct from heart failure, in which the pumping action of the heart is impaired. However, an MI may lead to heart failure.

Chest pain is the most common symptom of acute myocardial infarction and is often described as a sensation of tightness, pressure, or squeezing. Levine's sign, in which a person localizes the chest pain by clenching one or both fists over their sternum, has classically been thought to be predictive of cardiac chest pain, although a prospective observational study showed it had a poor positive predictive value.

Chest pain may be accompanied by sweating, nausea or vomiting, and fainting, and these symptoms may also occur without any pain at all. In women, the most common symptoms of myocardial infarction include shortness of breath, weakness, and fatigue.

In yet some further embodiments, the methods of the invention may be applicable for TTP. Thrombotic thrombocytopenic purpura (TTP) is a blood disorder that results in blood clots forming in small blood vessels throughout the body. This results in a low platelet count, low red blood cells due to their breakdown, and often kidneys, heart, and brain dysfunction. Symptoms may include large bruises, fever, weakness, shortness of breath, confusion, and headache. Repeated episodes may occur.

In about half of cases a trigger is identified, while in the remainder the cause remains unknown. Known triggers include bacterial infections, certain medications, autoimmune diseases such as lupus, and pregnancy. The underlying mechanism typically involves antibodies inhibiting the enzymeADAMTS-13. This results in decreased break down of large multimers of von Willebrand factor (vWF) into smaller units. Less commonly TTP is inherited from a person's parents, known as Upshaw-Schulman syndrome, such that ADAMTS-13 dysfunction is present from birth. Diagnosis is typically based on symptoms and blood tests. It may be supported by measuring activity of or antibodies against ADAMTS-13. With plasma exchange the risk of death has decreased from more than 90% to less than 20%. Immunosuppressants, such as glucocorticoids, and rituximabmay also be used.

Still further, in some embodiments, the methods of the invention may be applicable for DIC. Disseminated intravascular coagulation (DIC) is a condition in which blood clots form throughout the body, blocking small blood vessels. Symptoms may include chest pain, shortness of breath, leg pain, problems speaking, or problems moving parts of the body. As clotting factors and platelets are used up, bleeding may occur. This may include blood in the urine, blood in the stool, or bleeding into the skin. Complications may include organ failure.

Relatively common causes include sepsis, surgery, major trauma, cancer, and complications of pregnancy. Less common causes include snake bites, frostbite, and burns. There are two main types of DIC, acute (rapid onset) and chronic (slow onset). Diagnosis is typically based on blood tests. Findings may include low platelets, low fibrinogen, high INR, or high D-dimer. Treatment is mainly directed towards the underlying condition. Other measures may include giving platelets, cryoprecipitate, or fresh frozen plasma. Heparin may be useful in the slowly developing form.

In some further embodiments, the methods of the invention may be applicable for HUS. Hemolytic-uremic syndrome (HUS) is a group of blood disorders characterized by low red blood cells, acute kidney failure, and low platelets. Initial symptoms typically include bloody diarrhea, fever, vomiting, and weakness. Kidney problems and low platelets then occur as the diarrhea is improving. While children are more commonly affected, adults may have worse outcomes. Complications may include neurological problems and heart failure.

Most cases occur after infectious diarrhea due to a specific type of E. coli called O157:H7. Other causes include S. pneumoniae, Shigella, Salmonella, and certain medications. The underlying mechanism typically involves the production of Shiga toxin by the bacteria. Atypical hemolytic uremic syndrome (aHUS) is due to a genetic mutation and presents differently. Though both cause widespread inflammation and multiple blood clots in small blood vessels, a condition known as thrombotic microangiopathy.

Treatment involves supportive care and may include dialysis, steroids, blood transfusions, or plasmapheresis.

The early symptoms can include diarrhea (which is often bloody), stomach cramps, mild fever, or vomiting that results in dehydration and reduced urine. Related symptoms and signs include lethargy, decreased urine output, blood in the urine, kidney failure, low platelets, (which are needed for blood clotting), and destruction of red blood cells (microangiopathic hemolytic anemia). High blood pressure, jaundice (a yellow tinge in skin and the whites of the eyes), seizures, and bleeding into the skin can also occur.

Still further, in some embodiments, the methods disclosed herein may be applicable for cerebral infraction. A cerebral infarction is an area of necrotic tissue in the brain resulting from a blockage or narrowing in the arteries supplying blood and oxygen to the brain. The restricted oxygen due to the restricted blood supply causes an ischemic stroke that can result in an infarction, if the blood flow is not restored within a relatively short period of time. The blockage can be due to a thrombus, an embolus or an atheromatous stenosis of one or more arteries. Which arteries are problematic will determine which areas of the brain are affected (infarcted). These varying infarcts will produce different symptoms and outcomes.

Symptoms of cerebral infarction are determined by the parts of the brain affected. If the infarct is located in primary motor cortex, contralateral hemiparesis is said to occur. With brainstem localization, brainstem syndromes are typical: Wallenberg's syndrome, Weber's syndrome, Millard-Gubler syndrome, Benedikt syndrome or others. Infarctions will result in weakness and loss of sensation on the opposite side of the body. Physical examination of the head area will reveal abnormal pupil dilation, light reaction and lack of eye movement on opposite side. If the infarction occurs on the left side brain, speech will be slurred. Reflexes may be aggravated as well.

Still further, in some embodiments, the methods of the invention may be applicable for SLE. Systemic lupus erythematosus (SLE), also known simply as lupus, is an autoimmune disease in which the body's immune system mistakenly attacks healthy tissue in many parts of the body. Symptoms vary between people and may be mild to severe. Common symptoms include painful and swollen joints, fever, chest pain, hair loss, mouth ulcers, swollen lymph nodes, feeling tired, and a red rash which is most commonly on the face. Often there are periods of illness, called flares, and periods of remission during which there are few symptoms.

The cause of SLE is not clear. It is thought to involve genetics together with environmental factors. Among identical twins, if one is affected there is a 24% chance the other one will be as well. Female sex hormones, sunlight, smoking, vitamin D deficiency, and certain infections, are also believed to increase the risk. The mechanism involves an immune response by autoantibodies against a person's own tissues. These are most commonly anti-nuclear antibodies and they result in inflammation. Diagnosis can be difficult and is based on a combination of symptoms and laboratory tests. There are a number of other kinds of lupus erythematosus including discoid lupus erythematosus, neonatal lupus, and subacute cutaneous lupus erythematosus. There is no cure for SLE. Treatments may include NSAIDs, corticosteroids, immunosuppressants, hydroxychloroquine, and metotrexate. SLE significantly increases the risk of cardiovascular disease with this being the most common cause of death.

It should be understood that the preset disclosure, and specifically any of the methods and compositions disclosed herein are applicable for any of the disclosed coagulation-associated disorders as well as to any stage and grade of each of these conditions and symptoms thereof.

In some embodiments, the methods disclosed herein may further comprise the step of administering to the subject, a therapeutically effective amount of at least one tPA or any functional fragments or variants thereof or any composition thereof.

As discussed herein before and as exemplified by the present Examples, to further enhance the thrombolytic effect of the disclosed ADAMTS-13 mutants and variants, the present disclosure further provides a combined therapeutic approach, combining the use of fibrinolytic compounds such as tPA with the disclosed ADAMTS-13 variants and mutants. Thus, in some embodiments, the therapeutic and/or prophylactic methods disclosed herein may further comprise the step of administering to the treated subject, a therapeutically effective amount of at least one tPA or any functional fragments or variants thereof or any composition thereof. Alternatively, the combined treatment may be performed using a combined composition comprising both fibrinolytic compounds. Thus, in some further embodiments, the methods of the invention may comprise the step of administering to the subject, a therapeutically effective amount of a combined composition comprising a combination of at least one ADAMTS-13 mutant and/or any truncated variant thereof; and at least one tPA or any functional fragments or variants thereof. In some embodiments, the combined composition may be as defined above. Therefore, the invention further encompasses the option of combined therapy.

It should be further appreciated and understood that the mutants, compositions and methods disclosed herein may be combined with any other anti coagulating agent, when used for treating and/or preventing coagulation related conditions. The therapeutic anti-coagulating mutants may therefore enhance any anti-coagulating effect of any other anticoagulating agent such as heparin, Warfarin (Comadin), clexane and the like.

In another aspect, the invention relates to a method for the treatment, amelioration, inhibition or prophylaxis of a disease, disorder, or condition associated with coagulation in a subject in need thereof. In some embodiments, the methods disclosed herein may comprise the step of administering to a subject treated with at least one tissue plasminogen activator (tPA), or any mutant or variant thereof, any/or with any fibrinolytic compounds, a therapeutically effective amount of at least one ADAMTS-13 mutant/s and/or variant/s (e.g., any truncated variant thereof) or any composition comprising the mutant/s and/or variant/s. In some embodiments, the mutant/s and/or variant/s used by the disclosed methods may carry at least one mutation and display resistance and/or reduced sensitivity to cleavage and/or inactivation by at least one tPA, or any mutant or variant thereof. In some embodiments, the mutant may carry at least one of the mutation that substitute the Arg³¹² residue and/or any amino acid residue adjacent to the Arg³¹² of the wild type ADAMTS-13 with a charged amino acid residue. The wild type ADAMTS-13 may comprise the amino acid sequence as denoted by SEQ ID NO: 2, or any variants or derivatives thereof.

In certain embodiments, the mutant/s and/or variant/s suitable for the method of the invention may be as the ADAMTS-13 mutant/s and/or variant/s of the invention, specifically, any of the mutants described above, or any combinations and kit/s thereof. In some alternative or additional embodiments, the methods disclosed herein may comprise the step of administering to a subject treated with at least one serine protease, a therapeutically effective amount of at least one ADAMTS-13 mutant, any truncated variant thereof or any composition comprising the mutants of the invention.

In certain embodiments, the serine protease may be tPA, uPA, plasmin, thrombin or granulocyte elastase or other plasminogen activators like streptokinase or any functional fragments or variants thereof.

Still further, in some further embodiments, the method disclosed herein may comprise the step of administering to a subject treated with at least anti-coagulation compound (e.g., heparin, Warfarin (Comadin), clexan and the like), a therapeutically effective amount of at least one ADAMTS-13 mutant, any truncated variant thereof or any composition comprising the mutants of the invention.

In yet some other embodiments, the methods of the present disclosure may be applicable for any coagulation-related conditions as disclosed herein before. In yet some further embodiments, the disclosed methods may be applicable for any one of DVT, PE, AIS, AMI, TTP, DIC, HUS, and SLE.

Still further, as discussed herein, the presently disclosed method is based on combined treatment regimen wherein the ADAMTS-13 mutant/s and/or variant/s discussed herein are administered to a patient that is already treated with a fibrinolytic compound such as tPA. However, it should be appreciated that the present disclosure further provides methods where an affective amount of at least one fibrinolytic compound, for example tPA is administered to a subject treated with at least one of the ADAMTS-13 mutant/s and/or variant/s of the present disclosure.

It should be understood that in some embodiments, all components are administered simultaneously (e.g., at the same time) or consecutively (e.g., one right after the other). For example, at least one ADAMTS-13 mutant and/or variant/s as disclosed herein may be administered prior to, after and/or simultaneously with administration of the at least one fibrinolytic agent or compound, specifically, tPA. Thus, according to some embodiments, the methods of the invention may further encompass administering to the treated subject at least one ADAMTS-13 mutant and/or variant/s as disclosed herein, for example, the ADAMTS-13 R312K mutant and/or any variant thereof, prior to, after and/or simultaneously with administration of the at least one fibrinolytic agent or compound, specifically, tPA.

Still further, the present disclosure provides therapeutic methods and compositions that administer an effective amount of the ADAMTS-13 mutant/s and or variant/s disclosed herein, to a subject in need thereof. The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician). More specifically, the term “effective amount” relates to the amount of an active agent present in a composition, specifically, the ADAMTS-13 mutant/s and or variant/s of the invention as described herein that is needed to provide a desired level of active agent in the bloodstream or at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the ADAMTS-13 mutant/s and or variant/s of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

Still further, in some embodiments, an effective amount of the ADAMTS-13 mutant/s and or variant/s may range between about 0.1 to about 0.9 gr/day/kg. More specifically, between about 0.1 to about 0.2 gr per day/per kg, about 0.2 gr per day/per kg or between about 0.2 to about 0.3 gr per day/per kg, about 0.3 gr per day/per kg or between about 0.3 to about 0.4 gr per day/per kg, about 0.4 gr per day/per kg or between about 0.4 to about 0.5 gr per day/per kg, about 0.5 gr per day/per kg or between about 0.5 to about 0.6 gr per day/per kg, about 0.6 gr per day/per kg or between about 0.6 to about 0.7 gr per day/per kg, about 0.7 gr per day/per kg or between about 0.7 to about 0.8 gr per day/per kg, about 0.8 gr per day/per kg or between about 0.8 to about 0.9 gr per day/per kg, about 0.9 gr per day/per kg or between about 0.9 to about but no more than 0.99 gr per day/per kg, and in some embodiments, less than 1 gr per day/per kg, for each of the ADAMTS-13 mutant/s and or variant/s. In yet some further embodiments, an effective amount of the ADAMTS-13 mutant/s and or variant/s may range between about 0.1 to about 10 mg/day/kg. More specifically, between about 0.5 to about 8 mg per day/per kg, about 8 mg per day/per kg or between about 1 to about 7 mg per day/per kg, about 7 mg per day/per kg or between about 1.5 to about 6 mg per day/per kg, about 6 mg per day/per kg or between about 2 to about 5 mg per day/per kg, about 5 mg per day/per kg or between about 2.5 to about 4.5 mg per day/per kg, about 4.5 mg per day/per kg or between about 3 to about 4 mg per day/per kg, about 4 mg per day/per kg or between about 0.1 to about 1 mg per day/per kg, about 1 mg per day/per kg or between about 0.5 to about 0.9 mg per day/per kg, about 0.9 mg per day/per kg and in some embodiments, less than 10 mg per day/per kg, for each of the ADAMTS-13 mutant/s and or variant/s. In yet some particular and non-limiting embodiments, an effective amount of the ADAMTS-13 mutant/s and or variant/s is about 1 mg/kg/day. It should be appreciated however that the indicated effective doses per day, or dosage unit as discussed herein, may be given either in a single administration or in two or more administrations at several time-points over 24 hr. Still further, administration and doses are determined by good medical practice of the attending physician and may depend on various general conditions of the subject in need.

The invention provides therapeutic methods and compositions for treating specific pathologic conditions and disorders. As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention”, “prophylaxis” as used herein, includes the prevention or postponement of development of the disease, prevention or postponement of development of symptoms and/or a reduction in the severity of such symptoms that will or are expected to develop, preventing the occurrence or reoccurrence of the acute disease attacks. In yet some further embodiments, prophylaxis also encompasses any reduction or attenuation of the susceptibility to develop the disease, and any reduction or inhibition of the occurrence or reoccurrence of the disease. These further include ameliorating existing symptoms, preventing-additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated directly or indirectly with the coagulation, as described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a pathologic disorder or coagulation process and their symptoms slowing their progress, further exacerbation or development, so as to appear later than in the absence of the treatment according to the invention.

More specifically, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The present invention relates to the treatment of subjects, or patients, in need thereof. By “patient” or “subject in need” it is meant any organism to whom the preventive and prophylactic combinations, composition/s, kit/s, and methods herein described is desired, including humans and domestic and/or wild mammals. More specifically, the therapeutic methods disclosed herein are applicable for any subject. In some particular embodiments, the methods of the invention may be particularly applicable for a mammal (specifically, at least one of a human, Cattle, rodent, domestic pig (swine, hog), sheep, horse, goat, alpaca, lama and Camels), an avian, an insect, a fish, an amphibian, a reptile, a crustacean, a crab, a lobster, a snail, a clam, an octopus, a starfish, a sea-urchin, jellyfish, and worms, specifically, a mammalian subject. In some specific embodiments, the treated subject may be a human subject. The subject may be male or female, a child or an adult. In exemplary embodiments, the subject is an adult (e.g., at least 18 years old). The present invention relates to the treatment of subjects, or patients, in need thereof. It should be further noted that particularly in case of human subject, administering of the compositions of the invention to the patient includes both self-administration and administration to the patient by another person. It should be understood that any suitable administration mode may be applicable for the present disclosure, specifically, any systemic or local administration. Still further, any suitable route including intraperitoneal, subcutaneous, transcutaneous, topical, intramuscular, intraarticular, subconjunctival, or mucosal, e.g. oral, intranasal, or intraocular administration. Local administration to the area in need of treatment may be achieved by, for example, by local infusion during surgery, topical application, direct injection into the specific organ. More specifically, the ADAMTSTM-13 mutant/s and/or variant/s, as well as compositions and combined compositions thereof used in any of the methods of the invention, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

As noted above, the present invention may involve the use of different active ingredients specifically the ADAMTS-13 mutant of the invention and tPA, that may be administered through different routes, dosages and combinations. More specifically, the treatment of coagulation associated diseases with a combination of active ingredients may involve separate administration of each active ingredient. Therefore, a kit providing a convenient modular format for the combined therapy would allow the desired or preferred flexibility in the above parameters.

Thus, in another aspect, the invention relates to a kit comprising:

First (a), at least one ADAMTS-13 mutant/s and/or variant (e.g., truncated variant thereof), or any composition thereof, optionally, in at least one dosage form. In certain embodiment, the mutant may carry at least one mutation, and displays resistance and/or reduced sensitivity to cleavage and/or inactivation by at least one tPA, or any mutant or variant thereof.

The second component (b), may be at least one tPA or any functional fragments or variants thereof, or any composition thereof, optionally, in at least one second dosage form. It should be understood however, that the present disclosure further encompasses kits that comprise in addition to the ADAMTS-13 mutants disclosed herein, any other serin proteases as specified above, and/or any other anti-coagulating compounds (e.g., heparin, Warfarin (Comadin), clexan and the like).

In some embodiments, the mutant of the kits of the present disclosure carries at least one mutation that may substitute the Arg³¹² residue and/or any amino acid residue adjacent to the Arg³¹² of the wild type ADAMTS-13 with a charged amino acid residue. The wild type ADAMTS-13 may comprise the amino acid sequence as denoted by SEQ ID NO: 2.

In other embodiments, the mutant suitable for the kit of the invention may comprise substitution of residue 312, and/or any adjacent residue to any charged residue, specifically, any one of lysine, aspartic acid, glutamic acid or histidine. In some specific embodiments, the charged amino acid may be lysine.

In some more specific embodiment, the mutant/s and/or variant/s of the kit of the invention may carry a mutation substituting the Arginine in position 312 to lysine and may be designated R312K. In some embodiments, such mutant comprises the amino acid sequence as denoted by SEQ ID NO:11, or any derivatives or variants thereof. In yet some further embodiments, the mutant may be a truncated variant of the mutant of the invention, that may comprise in some embodiments, the amino acid sequence as denoted by SEQ ID NO:5, or any derivatives or variants thereof.

In yet some other embodiments, the mutant of the kit of the invention may be valine 313, and may carry a mutation substituting valine 313 with aspartic acid. In some embodiments, the mutant may be designated V313D. Still further, in some specific embodiments, such mutant may comprise the amino acid sequence as denoted by SEQ ID NO: 14, or any derivatives or variants thereof. In yet some further embodiments, a truncated variant of such mutant may comprise the amino acid sequence as denoted by SEQ ID NO: 17.

In some specific embodiments, any mutant/s or variant/s of ADAMTS-13 disclosed herein is suitable for the kits disclosed herein, with the proviso that the mutant and/or variant does not carry a mutation substituting the Arginine in position 312 to Alanine.

In some other embodiments, the kit of the invention may comprise: In a first component (a), at least one ADAMTS-13 mutant, any truncated variant thereof, or any composition thereof. In certain embodiment, the mutant may carry at least one mutation. More specifically, at least one of the mutations in the mutant of the invention may be the substitution of the Arg³¹² residue or any amino acid residue adjacent to the Arg³¹² of the wild type ADAMTS-13 with a charged amino acid residue. The wild type ADAMTS-13 may comprise the amino acid sequence as denoted by SEQ ID NO: 2. The second component of the kit of the invention (b), may comprise at least one serine protease or any functional fragments or variants thereof, or any composition thereof. In some further embodiments, the serine protease may be any one of tPA, uPA, plasmin, thrombin or granulocyte elastase or other plasminogen activators like streptokinase or any functional fragments or variants thereof.

According to some embodiments, the kit of the invention may further comprise container means for containing the different components of the kit of the invention or any dosage forms thereof. The term “container” as used herein refers to any receptacle capable of holding at least one component of a pharmaceutical composition of the invention. Such a container may be any jar, vial or box known to a person skilled in the art and may be made of any material suitable for the components contained therein and additionally suitable for short- or long-term storage under any kind of temperature. More specifically, the kit includes container means for containing separate composition/s; such as a divided bottle or a divided foil packet however, the separate compositions may also be contained within a single, undivided container. Typically, the kit includes directions for the administration of the separate components, compounds or agents. As noted above, the kit form is particularly advantageous when the separate components, compounds or agents are preferably administered in different dosage forms (e.g., oral and parenteral), are administered at different dosage intervals, or when titration of the individual components of the combination is desired by the prescribing physician.

In yet some other embodiments, the kit of the invention may be for use in a method for the treatment, amelioration, inhibition or prophylaxis of any disease, disorder, or condition associated with coagulation in a subject in need thereof. It should be further appreciated that the kits disclosed herein may be applicable for any of the disclosed pathologies.

In some specific and non-limiting embodiments, the kits of the present disclosure may be applicable for any one of DVT, PE, AIS, AMI, TTP, DIC, HUS, SLE.

In a further aspect, the invention relates to at least one ADAMTS-13 mutant, any truncated variant thereof or any composition comprising the mutant, for use in a method of treatment, amelioration, inhibition or prophylaxis of a disease, disorder, or condition associated with coagulation in a subject in need thereof. In some embodiments, the mutant may carry at least one mutation and displays resistance and/or reduced sensitivity to cleavage and/or inactivation by at least one tPA, or any mutant or variant thereof.

In some embodiments, the mutant carries at least one mutation that substitute the Arg³12 residue or any amino acid residue adjacent to the Arg³12 of the wild type ADAMTS-13 with a charged amino acid residue. It should be noted that the wild type ADAMTS-13 comprises the amino acid sequence as denoted by SEQ ID NO: 2.

In some further embodiments, the charged amino acid residue of the at least one ADAMTS-13 mutant for use according to the invention, may be any one of lysine, aspartic acid, glutamic acid or histidine.

In some specific embodiments, the charged amino acid residue of the at least one ADAMTS-13 mutant for use according to the invention, may be lysine.

In some more specific embodiments, the at least one ADAMTS-13 mutant for use according to the invention, may carry a mutation substituting the Arginine in position 312 to lysine and may be designated R312K. Still further, in some embodiments, the mutant may comprise the amino acid sequence as denoted by SEQ ID NO:11 or any derivatives or variants thereof. In yet some further embodiments, the truncated variant of the mutant may comprise the amino acid sequence as denoted by SEQ ID NO:5 or any derivatives or variants thereof.

In yet another embodiment, the mutant for use according to the invention may be valine 313 and may carry a mutation substituting valine 313 with aspartic acid. In some embodiments, the mutant may be designated V313D. In some specific embodiments, such mutant may comprise the amino acid sequence as denoted by SEQ ID NO: 14, or any derivatives or variants thereof. In yet some further embodiments, a truncated version of such mutant may carry the amino acid sequence as denoted by SEQ ID NO: 17, or any derivatives or variants thereof.

In some further embodiments, the at least one ADAMTS-13 mutant for use according to the invention, may be as described above.

In some embodiments, the at least one ADAMTS-13 mutant for use according to the invention, may display resistance and/or decreased sensitivity to cleavage and/or inactivation by at least one tPA, or any mutant or variant thereof.

In some embodiments, the at least one ADAMTS-13 mutant for use according to the invention, may display resistance to cleavage by at least one serine protease or variant thereof. In certain embodiments, the serine protease may be tPA, uPA, plasmin, thrombin or granulocyte elastase or other plasminogen activators like streptokinase or any functional fragments or variants thereof.

In some further embodiments, the at least one ADAMTS-13 mutant for use according to the invention, refers to a mutant or any truncated variant thereof that display an increased activity.

In yet some further embodiments, the ADAMTS-13 mutant for use in accordance with the present disclosure may display a prolonged half-life relative to ADAMTS-13 wild type.

In yet some other embodiments, the at least one ADAMTS-13 mutant for use according to the invention, may be relevant to any disease, disorder, or condition associated with coagulation. In some particular and non-limiting embodiments, such disorders may be any one of DVT, PE, AIS, AMI, TTP, DIC, HUS, SLE.

Still further, the invention provides a therapeutically effective amount of at least one ADAMTS-13 mutant/s and/or variant/s (e.g., any truncated variant thereof) or any composition comprising the mutant for use in a method for the treatment, amelioration, inhibition or prophylaxis of a disease, disorder, or condition associated with coagulation in a subject in need thereof. It should be noted that the subject is a subject being treated with at least one tPA, or any mutant or variant thereof. In yet some further specific embodiments, the mutant carries at least one mutation, and displays resistance and/or reduced sensitivity to cleavage and/or inactivation by at least one tPA, or any mutant or variant thereof. In some embodiments, the mutant carries at least one mutation that in more specific embodiments, substitutes the Arg³¹² residue and/or any amino acid residue adjacent to said Arg³¹² of the wild type ADAMTS-13 with a charged amino acid residue.

In some embodiments, the ADAMTS-13 mutant provided for use in accordance with the invention is any of the mutants disclosed by the invention.

In yet some further embodiments, the use according to the invention is specifically applicable for subjects suffering from at least one disease, disorder, or condition, specifically, any disease or disorder associates with coagulation. In some embodiments, such disorders may be any one of DVT, PE, AIS, AMI, TTP, DIC, HUS, SLE.

Still further, in another aspect, the invention relates to a mutant of ADAMTS-13 that carries at least one mutation. In some embodiments, at least one of the mutations in the mutants of the invention may substitute the Arginine in position 312 (Arg312) or in any amino acid residue adjacent to the Arg312 of the wild type ADAMTS-13 with a charged amino acid residue, or any truncated variant thereof. It should be understood that residue 312, as indicated herein refers to the amino acid sequence of the wild type ADAMTS-13, that may comprise the amino acid sequence as denoted by SEQ ID NO: 2, or any variants or derivatives thereof.

In yet some other aspect, the invention relates to a composition comprising an effective amount of at least one ADAMTS-13 mutant, or any truncated variant thereof. In some embodiments, the mutant may carry a mutation and at least one of the mutation/s may substitute the Arg³¹² residue or any amino acid residue adjacent to the Arg³¹² of the wild type ADAMTS-13 with a charged amino acid residue. It should be noted that the wild type ADAMTS-13 comprises the amino acid sequence as denoted by SEQ ID NO: 2, and therefore the position 312, refers to SEQ ID NO: 2. In some embodiments, the composition may optionally further comprise at least one pharmaceutically acceptable carrier, diluent, excipient and/or additive.

In another aspect, the invention relates to a combined composition comprising a combination of at least one ADAMTS-13 mutant or any truncated variant thereof, and at least one tPA or any functional fragments or variants thereof. In some embodiments, the mutant may carry at least one mutation. The at least one of the mutation/s may substitute the Arg³¹² residue or any amino acid residue adjacent to said Arg³¹² of the wild type ADAMTS-13 with a charged amino acid residue. It should be noted that the wild type ADAMTS-13 may comprise the amino acid sequence as denoted by SEQ ID NO: 2, or any variants or derivatives thereof. Still further, in some embodiments, the composition may optionally further comprise at least one pharmaceutically acceptable carrier, diluent, excipient and/or additive.

In a further aspect, the invention relates to a method for the treatment, amelioration, inhibition or prophylaxis of a disease, disorder, or condition associated with coagulation in a subject in need thereof. In some embodiments, the method may comprise the step of administering to the subject a therapeutically effective amount of at least one ADAMTS-13 mutant, any truncated variant thereof, or any composition or combined composition comprising the mutant of the invention. In certain embodiments, the mutants of the invention may carry at least one mutation. More specifically, at least one of the mutation/s may substitute the Arg312 residue or any amino acid residue adjacent to the Arg312 of the wild type ADAMTS-13 with a charged amino acid residue. It should be noted that the wild type ADAMTS-13 may comprise the amino acid sequence as denoted by SEQ ID NO: 2.

In another aspect, the invention relates to a method for the treatment, amelioration, inhibition or prophylaxis of a disease, disorder, or condition associated with coagulation in a subject in need thereof, comprising the step of administering to a subject treated with at least one tissue plasminogen activator (tPA), or any mutant or variant thereof, a therapeutically effective amount of at least one ADAMTS-13 mutant, any truncated variant thereof or any composition comprising the mutant. In some embodiments, the mutant may carry a mutation, at least one of the mutation/s may substitute the Arg³12 residue or any amino acid residue adjacent to said Arg³¹² of the wild type ADAMTS-13 with a charged amino acid residue. The wild type ADAMTS-13 may comprise the amino acid sequence as denoted by SEQ ID NO: 2, or any variants or derivatives thereof.

In yet another aspect, the invention relates to a kit comprising: First (a), at least one ADAMTS-13 mutant, any truncated variant thereof, or any composition thereof. In certain embodiment, the mutant may carry at least one mutation, at least one of the mutation/s may substitute the Arg³¹² residue or any amino acid residue adjacent to the Arg³¹² of the wild type ADAMTS-13 with a charged amino acid residue. The said wild type ADAMTS-13 may comprise the amino acid sequence as denoted by SEQ ID NO: 2. The second component (b), may be at least one tPA or any functional fragments or variants thereof, or any composition thereof.

In a further aspect, the invention relates to at least one ADAMTS-13 mutant, any truncated variant thereof or any composition comprising the mutant, for use in a method of treatment, amelioration, inhibition or prophylaxis of a disease, disorder, or condition associated with coagulation in a subject in need thereof. In some embodiments, the mutant may carry at least one mutation. More specifically, at least one of the mutation/s may substitute the Arg³¹² residue or any amino acid residue adjacent to the Arg³12 of the wild type ADAMTS-13 with a charged amino acid residue. It should be noted that the wild type ADAMTS-13 comprises the amino acid sequence as denoted by SEQ ID NO: 2.

The invention further provides a therapeutically effective amount of at least one ADAMTS-13 mutant, any truncated variant thereof or any composition comprising the mutant for use in a method for the treatment, amelioration, inhibition or prophylaxis of a disease, disorder, or condition associated with coagulation in a subject in need thereof. It should be noted that the subject is a subject being treated with at least one tPA, or any mutant or variant thereof. In yet some further specific embodiments, the mutant carries at least one mutation, in more specific embodiments, the mutation substitutes the Arg³12 residue or any amino acid residue adjacent to said Arg³¹² of the wild type ADAMTS-13 with a charged amino acid residue.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Experimental Procedures

Expression of MDTCS R312K in S2 Cells with pMT Based Plasmid and Protein Purification

The plasmid had been transfected into S2 cells, and the cells were selected with hygromycin B. The growing media was Schneider's Drosophila Medium containing 10% of FBS, 0.5% of P/S (optional), 0.2 mg/ml of hygromycin B. The expression media was Insect Xpress from. The S2 cells were grown at room temperature, (best temperature is 26° C.). 1 vial (tube) of stock S2 cells is taken with expected plasmid transfected from liquid N2. The cells were suspended and moved into a T25 flask. 5 ml (or 4 ml) of growing media was added with repeated pipetting. The cells were incubated at room temperature overnight. On the next day, change the fresh growing media is changed and the cells were incubated at room temperature for 3 day (±1 day) until the cell start to clump. The cells were suspended by pipette, then moved into a 15-ml tube, spin down with 1000 rpm for 5 min, and the supernatant was aspirated away. The cells were re-suspended the cells 5 ml of fresh media, moved into a T75 flask and media was added to final 15-20 ml. Incubate at room temperature. The cell density was counted, based on the total volume. When the total cell number was 400×10⁶ or higher, the cells were spin down the cells. The cells were re-suspended with 100 ml of Insect-Xpress media (Pen/Strep added) and moved into a PETG flask. The cell density was checked daily. When the cell density was or higher than 20×10⁶ cells/ml, fresh Xpress media was added to final 500 ml (400-500 ml), The final cell density was 4×10⁶ cells/ml or higher and the cells were shake. At same day, after 2-3 hrs shake, CuSO4 was added to 0.5 mM and cells were shake. Cell density was checked, when it was higher than 30×10⁶ cells/ml (3-5 days), the cells were spin down with 2000 rpm for 10 min, the supernatant was collected. A filter with 0.2 μm filter Unit was used.

At the C-terminus, the DMTCS R311K construct has a triple-FLAG tag. For purification, the supernatant was loaded on an anti-FLAG column (commercially available). The column was balanced with either Insect Xpress media or PBS before loading the sample. The loading speed was determined by natural flow, usually loading no more than 100-200 ml for an hour to a few hours at cold room. Wash column with PBS, run 10× column volume. The sample was eluted either with 0.1M Glycine-HCl pH 2.6-2.8 and neutralized with ⅛ volume of Tris-HCl pH 8.0, or in accordance with the column supplier recommendations. The neutralized MDTCS R311K protein was collected and concentrated with the simultaneous buffer change for PBS on Amicon Ultra Centrifugal Filters 50K. The protein concentration was checked at OD₂₈₀ 1.186=1 mg/ml.

Construction of the Truncated R312K Mutant:

The mutated R312K truncated sequence starts from amino acid residue 75 in the mature ADAMTS-13 sequence (first 74 amino acids cut) and ends at the amino acid residue 685 of the mature ADAMTS-13 sequence (see amino acid sequence as denoted by SEQ ID NO: 5). The WT truncated ADAMTS comprise an amino acid sequence as denoted by SEQ ID NO: 7, encoded by the nucleic acid sequence as denoted by SEQ ID NO: 6. In order to construct the truncated R312K mutant the following primers were used: the forward primer as denoted by SEQ ID NO: 8 and the reverse primer as denoted by SEQ ID NO: 9, as well as the AntiK primer as denoted by SEQ ID NO: 10. Primer SEQ ID NO: 20 was used to mutate R312 to A.

### Example 1

tPA Cleaves and Inactivated ADAMTS-13

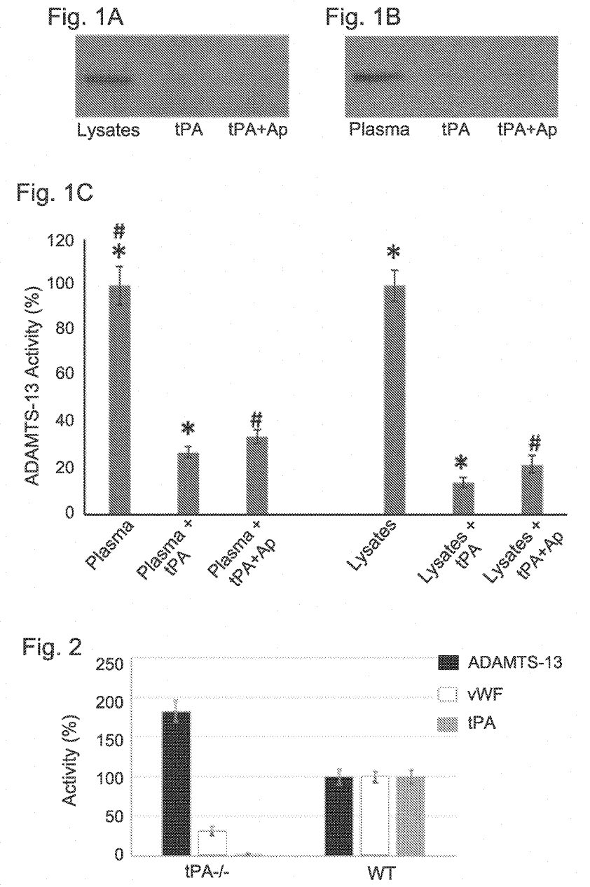

To examine the capacity of tPA to cleave ADAMTS-13, fetal liver cells lysates (A549 cells) and human plasma which express the native ADAMTS-13 were incubated in the absence or presence of tPA (100 nM) for 2 hrs at 37° C. To exclude the possibility that tPA cleaves ADAMTS-13 via plasminogen activation to plasmin, the lysates and plasma were incubated in tPA with the serine protease—aprotinin—a plasmin inhibitor (1 μM). Western blots were performed using monoclonal anti-ADAMTS-13 antibody EPR6132 from Abcam. FIG. 1 A-B show that tPA cleaves ADAMTS-13 in cell lysates (A) and human plasma (B), and the cleavage is unaffected by the plasmin inhibitor (tPA+Ap), indicating that ADMATS-13 is not cleaved by plasmin, and most likely, cleaved by tPA. To investigate the physiological relevance of these observations, it was asked whether tPA regulates ADAMTS-13 activity in vitro and in vivo and whether the tPA ADAMTS-13 interactions affects vWF activity in vivo. The ADAMTS-13 activity was measured in cell lysates and human plasma, in the presence and absence of tPA (100 nM). Indeed, it was shown that ADAMTS-13 activity is significantly decreased in the presence of tPA, and the decrease in the ADAMTS-13 activity was unaffected by aprotonin (FIG. 1C). The ADAMTS-13 and vWF activities were further measured in WT and tPA−/− mice. Plasma ADAMTS-13 activity measured by ELISA was significantly higher (p<0.05) and vWF activity was lower (p<0.05) in tPA−/− mice (FIG. 2).

The data presented in FIGS. 1 and 2 support the contention that, under physiological conditions, tPA cleaves and inactivate ADAMTS-13 and subsequently increases vWF activity.

### Example 2

Regulation of ADAMTS-13 Activity

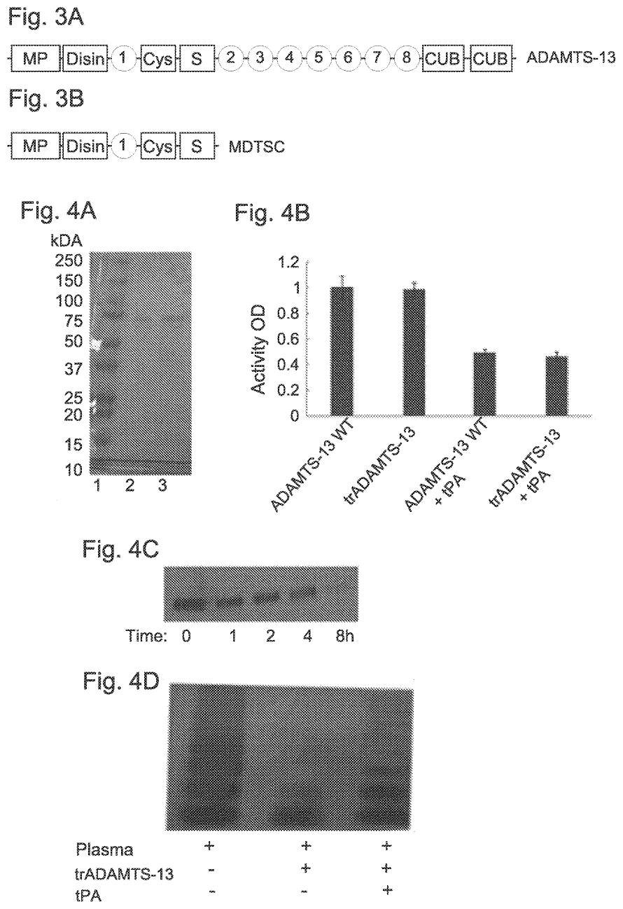

Human ADAMTS-13 is a multidomain protein (FIG. 3A) with Metalloprotease domain (MP), Disinterring-like domain (Disin), 8 Thrombospondin type-1 domains (1-8), Cysteine-rich domain (Cys), Spacer domain (S) and 2 CUB domains. The metalloprotease domain contains the catalytic site of ADAMTS-13 that cleaves vWF. Since it appears that tPA inhibits the catalytic activity of ADAMTS-13, it suggests that it affects its N terminal domains, that contains the metalloprotease domain where the catalytic activity is performed.

A truncated form of trADAMTS-13 containing the MP (M), Disin (D), 8 Thrombospondin (T), Cys (C), (S) domains, indicated herein as MDTCS subunits was therefore synthetized, creating a recombinant truncated ADAMTS-13 variant (trADAMTS-13), that contains MP domain (FIG. 3B).

trADAMTS-13 has the expected MW, of about 75 KDa (FIG. 4A), is fully active as the ADAMTS-13 WT (FIG. 4B), and in the presence of tPA, it is cleaved (FIG. 4C). As expected, the activity of both trADAMTS-13 and ADAMTS-13 WT was reduced in the presence of tPA (FIG. 4B). Similarly, the inactivation of trADAMTS-13 by tPA prevented the proteolysis of ULvWV multimers present in human plasma (FIG. 4D), indicating that trADAMTS-13 cleaves vWF and that tPA prevents such an effect. These results clearly demonstrate that the truncated ADAMTS-13 variant retains its ability to proteolytically inactivate ULvWF, and moreover, it retains the sensitivity to cleavage and/or inactivation by tPA.

### Example 3

Delineation of the ADAMTS-13 Cleavage Site

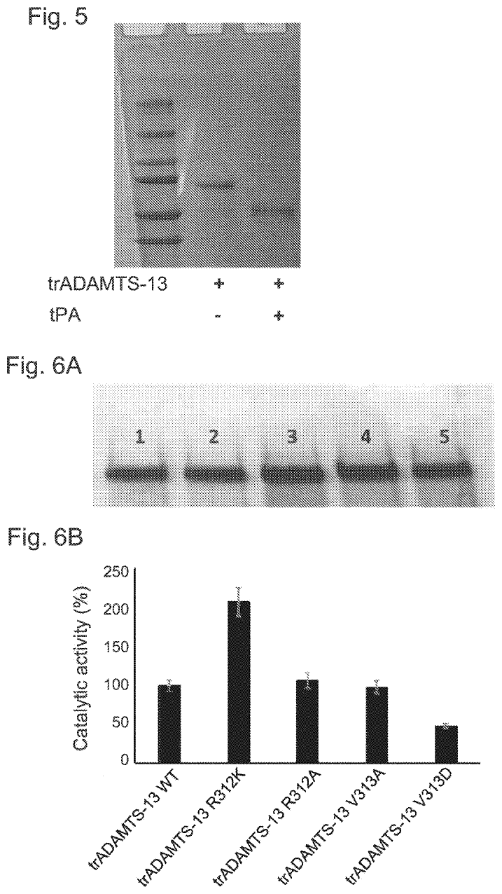

Mass spectroscopy was used to identify the amide bond in ADAMTS-13 cleaved by tPA. trADAMTS-13 was incubated with tPA, the degradation products were separated on SDS-PAGE (FIG. 5), individual bands were excised and their amino acid sequences were determined. Analysis of the sequence showed that tPA cleaves ADAMTS-13 at R³¹²-V³¹³, analogous to its cleavage site in plasminogen (Blood Coagul Fibrinolysis. 1992 3(5): 605-614).

### Example 4

Creation of variants that perturbs the R³¹²V³¹³ bond in ADAMTS-13

Based on the results presented above, the inventors hypothesized that a single mutation that perturbs the R³¹²-V³¹³ bond in ADAMTS-13 would produce a variant that would be resistant to cleavage by tPA. Such mutant is expected to retain its ability to cleave vWF and by that, to improve the trADAMTS-13 in-vivo activity.

To test this hypothesis, truncated recombinant ADAMTS-13 variants (trADAMTS-13) were next synthetized, with a single mutation where R312 was replaced by: (1) Lysine (K) (trADAMTS-13^{R312K}), (2) Alanine (A) (trADAMTS-13^{R312A}) or (3) by replacing V³¹³ with Alanine A (trADAMTS-13^{V313A}) or (4) with Aspartate (D) (trADAMTS-13^{V313D}) All the variants were cloned and expressed in S2 cells.

All the variants were analyzed by SDS-PAGE (FIG. 6A) and their catalytic activity on vWF was compared (FIG. 6B).

As shown by FIG. 6B, although all of the four variants migrate as a single chain molecule at the same Molecular Weight of the un-mutated variant (FIG. 6A), they exhibited different levels of catalytic activity (FIG. 6B).

trADAMTS-13^{R312K} was the most potent variant exhibiting enhanced proteolytic activity on vWF (FIG. 6B), in comparison with the un-mutated protein trADAMTS-13 WT. In line with the data presented by European patent application EP 2172544 [18], trADAMTS-13^{R312A} catalytic activity was not affected significantly by replacing R³¹² with A (FIG. 6B). Replacing V³¹³ with A (trADAMTS-13^{V313A}), also had no significant effect on its proteolytic activity on vWF (FIG. 6B). In contrast, replacing V³¹³ with D (trADAMTS-13^{V313D}) decreased by more than 50% the proteolytic activity of the resultant ADAMTS-13 variant on vWF (FIG. 6B).

However, surprisingly, substitution to lysine in position 312, led to loss of sensitivity to tPA. Specifically, trADAMTS-13^{R312K} was resistant to cleavage by tPA under conditions where trADAMTS-13 was proteolyzed completely (FIG. 7A) and lost its catalytic activity (FIG. 7B).

The half-life of the truncated forms of ADAMTS-13 WT and mutant were measured. More specifically, trADAMTS-13 WT or trADAMTS-13^{R312K} (1 mg in saline) were injected IP to WT mice (n=245 in each group). Each group of mice was divided into five sub-groups (n=5 in each group). Animals were scarified at 5, 30, 60, 180 and 360 minutes after injection, and ADAMTS-13 activity was determined. A group of five mice injected with saline was used as a control determining time 0. The inventors found that the t½ of the trADAMTS-13 was 132.8±41.3 min and that of trADAMTS-13^{R312K} was 344.6±131.1 min. Thus, the half-life of truncated ADAMTS-13 mutant is significantly longer than the truncated WT or the full-length WT molecules. These data indicate that the truncated variant display an extended activity time and is therefore suitable for events like DVT as well as from ischemic stroke.

Examining the effect of tPA on the catalytic activity of the 4 trADAMTS-13 variants, revealed that trADAMTS-13^{R312K} and trADAMTS-13^{V313D} variants were resistant to inactivation by tPA. In contrast, tPA inhibited the proteolytic activity of trADAMTS-13^{R312A} and trADAMTS-13^{V313A} variants on vWF (FIG. 8).

In summary, mutations in the R³¹²-V³¹³ site clearly affect the catalytic activity of ADAMTS-13. Although all of the tested variants maintained catalytic activity on vWF, trADAMTS-13^{R312K} variant is the most potent and is totally resistant to inactivation by tPA.

### Example 5

Effect of Autoantibodies from TTP Plasma on trADAMTS-13^{R312K} Activity

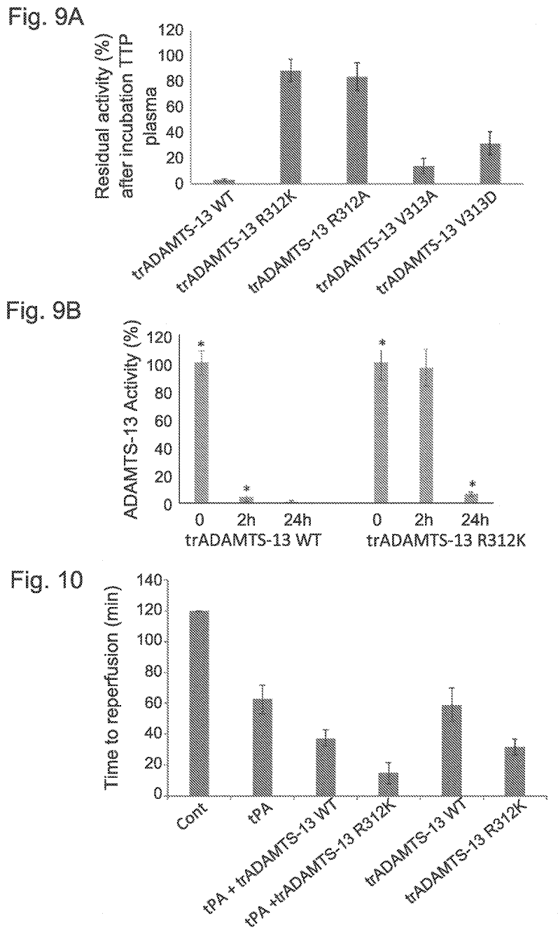

Anti-ADAMTS-13 autoantibodies cause severe enzyme deficiency. ADAMTS-13 deficiency causes the loss of regulation of vWF multimeric size and platelet function, which results in the formation of disseminated microvascular platelet microthrombi. Anti-ADAMTS-13 autoantibodies recognize mainly the N-terminal domains of the enzyme (Thomas et al. EBioMedicine 2015 2, 942-952), domains that are included in trADAMTS-13. R312 was shown to be one of the targets of the anti ADAMTS-13 antibodies (European patent application EP2172544 [18]). Furthermore, this publication indicates that only replacement with uncharged amino acid could reduce the antigenicity of ADAMTS-13 [18]. To examine the effect of anti ADAMTS-13 autoantibodies on the catalytic activity of the above described trADAMTS-13 variants, trADAMTS-13 WT and all of the four variants of the invention were incubated with plasma from patients with acquired ADAMTS-13 deficiency and <10% ADAMTS-13 activity for 2 hours. As expected, TTP plasma totally neutralized trADAMTS-13 WT. Surprisingly, trADAMTS-13^{R312K} was resistant to anti ADAMTS-13 autoantibodies (FIG. 9A-9B). The substantial resistance to inhibition of trADAMTS-13^{R312K} was shown around 2 hours up to 24 hours following incubation with TTP plasma (FIG. 9B). In addition, trADAMTS-13^{R312A} also appeared to be resistant to anti ADAMTS-13 autoantibodies. In contrast, trADAMTS-13V³13^{A} and trADAMTS-13V³13^{D} maintained their susceptibility to inhibition by the anti ADAMTS-13 autoantibodies (FIG. 9A).

### Example 6

Thrombolytic Effect of trADAMTS-13 Variants on VWF-Rich Thrombus

To test the therapeutic potential of ADAMTS-13 variants, a mouse model that recapitulates acute ischemic via thrombotic occlusion of the carotid artery after topical application of FeCl3, was next used. Histological analysis of the occlusive thrombi formed by topical application of FeCl3 demonstrated that they are rich in VWF (Denorme et al. Blood 2016 127:2337-2345).

As previously reported (Denorme et al. Blood 2016 127:2337-2345), thrombus formation was initiated by topical application of FeCl3 using a larger filter paper (0.53-1.5 mm) saturated with 20% FeCl3.

Sixteen weeks old C57B/6 mice were subjected to carotid artery occlusion. The left common carotid artery was isolated, and a vascular flow probe (Transonic Systems, NY) was applied to monitor blood flow. FeCl3 was applied as reported (Denorme et al. Blood 2016 127:2337-2345). The time required to form an occlusive thrombus, is defined as the time required to drop blood flow below 25%, after FeCl3 application. Five minutes after occlusion, mice were given intravenous injection of tPA (0.5 mg/kg) alone or together with increasing doses of ADAMTS-13 variants, trADAMTS-13 WT or trADAMTS-13^{R312K} (0.5, 1 or 5 mg/kg each), and blood flow restoration was monitored for 120 minus. In the untreated group (control), no improvement in blood flow was detected in blood flow even after 120 minutes of monitoring. FIG. 10 shows that time to reperfusion, was improved significantly by the co-administration of tPA and trADAMTS-13. FIG. 10 also shows that trADAMTS-13^{R312K} was more effective than the un-mutated variant. Moreover, this figure shows synergistic activity when combining tPA with the ADAMTS-13 mutant of the invention.

### Example 7

Delineation of the Anti-Thrombotic Activity of trADAMTS-13 In Vivo

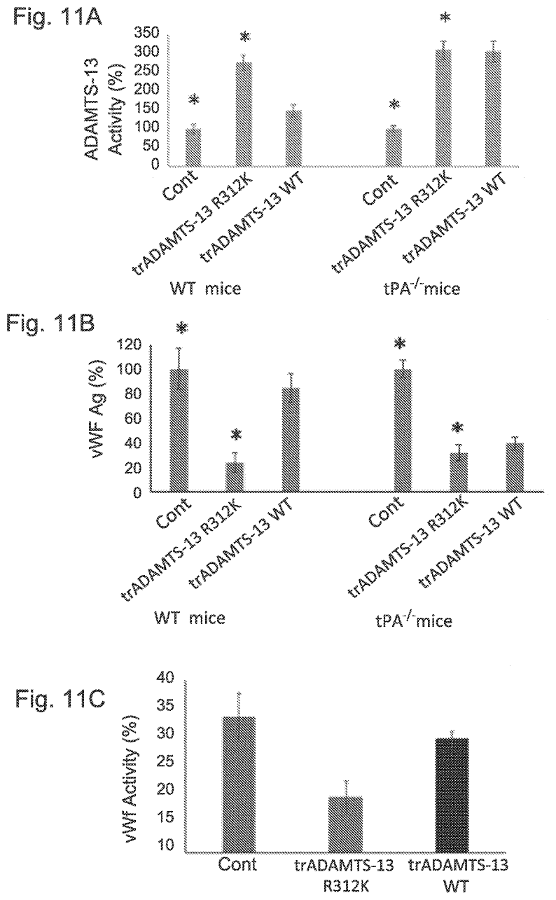

Since it appears that the anti-vWF activity of ADAMTS-13 is limited by tPA, it was hypothesized that by overcoming such limitation ADAMTS-13 thrombolytic/anti-coagulation activities would be improved. To test this hypothesis in in vivo setting, three sets of experiments were performed. First, the effect of tPA on ADAMTS-13 activity was examined followed by vWF concentration and activity. WT and tPA−/− mice were injected IP with trADAMTS-13 WT or trADAMTS-13^{R312K}. Two hours later, plasma ADAMTS-13 activity and concentration of vWF were assessed. Mice injected with trADAMTS-13^{R312K} retained greater capacity to proteolyze vWF than those given trADAMTS-13 WT (FIGS. 11A and 11C) and respectively, the concentration of vWF in trADAMTS-13^{R312K} was lower as well (FIG. 11B). The activity of the variant trADAMTS-13^{R312K} and in correlation the vWF weight were substantial similar in both tPA−/− mice and WT mice, whereas the activity of trADAMTS-13 WT was higher in the tPA−/− mice compared to WT mice (FIGS. 11A and 11B). Second, the effect of tPA resistance of ADAMTS-13 on the formation of venous clots was examined using an inferior vena cava (IVC) stasis model. WT mice were given an IP injection of trADAMTS-13 WT or trADAMTS-13^{R312K} one hour before inducing clot formation. trADAMTS-13^{R312K} reduced clot size to a significantly greater extent than trADAMTS-13 (FIG. 12B, p<0.05). Third, the effect on bleeding two hours after IP of trADAMTS-13 WT or tPA-resistant trADAMTS-13^{R312K} was examined in the tail cut model bleeding (Abu-Fanne et al. Blood. 2019 Jan. 31; 133(5):481-493). Although both truncated, ADAMTS-13 variants increased the bleeding time, trADAMTS-13^{R312K} caused a greater prolongation in the bleeding time than trADAMTS-13 WT (FIG. 13).

### Example 8

Soluble Fibrin Reverses tPA-Mediated Inactivation of trADAMTS-13

tPA is a well-established fibrinolytic protein. However, the surprising results showing that tPA-mediate ADAMTS-13 inactivation, raised the question whether there may be a potential procoagulant function of tPA.

It is also well-established the that the plasminogen activator activity of tPA is almost undetectable in the absence of fibrin (Hoylaerts M, Rijken D C, Linjen H R, Collen D 1982, Journal of Biological Chemistry, 257, 2912-2919). Therefore, to examine how these two seemingly opposing activities are regulated, the effect of soluble fibrin was examined on tPA-mediated inactivation of trADAMTS-13. The activity of trADAMTS-13 WT was measured following incubation for 4 hr in soluble fibrin or PBS, without or with tPA. The addition of soluble fibrin reversed the tPA-mediated reduction in ADAMTS-13 activity (FIG. 14A). Similarly, to measure the activity of wild type full length ADAMTS-13 in the presence of fibrin and tPA, serum from WT mice was incubated with tPA without or with fibrin. Similarly, the addition of fibrin, reversed the tPA-mediated inactivation in ADAMTS-13 activity (FIG. 14B).

These results show that fibrin down-regulates the potentially procoagulant role and upregulates the fibrinolytic actions of tPA.

1-59. (canceled)

60. A mutant and/or variant of a disintegrin and metalloproteinase with a thrombospondin type 1 motif, member 13 (ADAMTS-13) that carries at least one mutation, or any truncated variant thereof, wherein said mutant displays resistance and/or reduced sensitivity to cleavage and/or inactivation by at least one tissue plasminogen activator (tPA), or any mutant or variant thereof.

61. The ADAMTS-13 mutant and/or variant according to claim 60, wherein said mutant, variant or any truncated variant carries at least one mutation that substitutes the Arginine in position 312 (Arg312) of the wild type ADAMTS-13 and/or any amino acid residue adjacent to said Arg312, with a charged amino acid residue, optionally, wherein said charged amino acid residue is any one of lysine, aspartic acid, glutamic acid or histidine.

62. The ADAMTS-13 mutant and/or variant, according to claim 60, or any truncated variant thereof, wherein said mutant carries at least one mutation that substitutes the Arginine in position 312 to lysine, is designated R312K, and wherein at least one of:
(a) said mutant comprises the amino acid sequence as denoted by SEQ ID NO:11, or any derivatives or variants thereof; and
(b) said truncated variant of said mutant comprises the amino acid sequence as denoted by SEQ ID NO:5, or any derivatives or variants thereof, and optionally, wherein said adjacent amino acid residue is valine 313, optionally, said mutant carries a mutation that substitutes valine 313 with aspartic acid.

63. The ADAMTS-13 mutant and/or variant according to claim 60, wherein said mutant or any truncated variant thereof display an increased activity, optionally, said mutant or any truncated variant thereof display a prolonged half-life relative to ADAMTS-13 wild type.

64. A composition comprising an effective amount of the at least one ADAMTS-13 mutant according to claim 60, and/or variant that carries at least one mutation, said composition optionally further comprises at least one pharmaceutically acceptable carrier, diluent, excipient and/or additive.

65. A combined composition comprising a combination of at least one ADAMTS-13 mutant according to claim 60, and/or variant and at least one tPA or any functional fragments or variants thereof, wherein said mutant, variant or any truncated variant thereof, carries at least one mutation and displays resistance and/or reduced sensitivity to cleavage and/or inactivation by at least one tPA, or any mutant or variant thereof.

66. The combined composition according to claim 65, wherein said composition further comprises fibrin.

67. A method for the treatment, amelioration, inhibition or prophylaxis of a disease, disorder, or condition associated with coagulation in a subject in need thereof, the method comprising the step of administering to said subject a therapeutically effective amount of at least one ADAMTS-13 mutant and/or variant, or any truncated variant thereof, or any composition or combined composition comprising said mutant, wherein said mutant carries at least one mutation and displays resistance and/or reduced sensitivity to cleavage and/or inactivation, by at least one tPA, or any mutant or variant thereof.

68. The method according to claim 67, wherein said mutant and/or variant or any truncated variant thereof carries at least one mutation that substitutes the Arg312 residue of the wild type ADAMTS-13, and/or any amino acid residue adjacent to said Arg312, with a charged amino acid residue, optionally, said charged amino acid residue is any one of lysine, aspartic acid, glutamic acid or histidine.

69. The method according to claim 67, wherein said mutant, variant or any truncated variant thereof carries a mutation substituting the Arginine in position 312 to lysine and is designated R312K, wherein at least one of:
(a) said mutant comprises the amino acid sequence as denoted by SEQ ID NO:11, or any derivatives or variants thereof, and
(b) said truncated variant of said mutant comprises the amino acid sequence as denoted by SEQ ID NO:5, or any derivatives or variants thereof, optionally, said adjacent amino acid reside is valine 313, optionally, said mutant, variant, or any truncated variant thereof carries a mutation that substitutes valine 313 with aspartic acid.

70. The method according to claim 67, wherein at least one of:
(a) said mutant, variant or any truncated variant thereof or any truncated variant thereof display an increased activity;
(b) said mutant, variant or any truncated variant thereof or any truncated variant thereof displays a prolonged half-life relative to ADAMTS-13 wild type; and
(c) wherein said disease, disorder, or condition is at least one of deep venous thrombosis (DVT), pulmonary emboli (PE), acute ischemic stroke (AIS), acute myocardial function (AMI), thrombotic thrombocytopenic purpura (TTP), disseminated intravascular coagulation (DIC), hemolytic-uremic syndrome (HUS), cerebral infarction or systemic lupus erythematosus (SLE).

71. The method according to claim 67, wherein:
(a) said method further comprises the step of administering to said subject, a therapeutically effective amount of at least one tPA or any functional fragments or variants thereof or any composition thereof; or
(b) said method comprising the step of administering to said subject, a therapeutically effective amount of a combined composition comprising a combination of at least one ADAMTS-13 mutant and/or any truncated variant thereof, and at least one tPA or any functional fragments or variants thereof.

72. The method according to claim 67, wherein said subject is a subject treated with at least one tPA, or any mutant, variant or any truncated variant thereof, a therapeutically effective amount of at least one ADAMTS-13 mutant, any truncated variant thereof or any composition comprising said mutant, wherein said mutant carries at least one mutation and displays resistance and/or reduced sensitivity to cleavage and/or inactivation by at least one tPA, or any mutant or variant thereof, optionally, wherein said disease, disorder, or condition is any one of DVT, PE, AIS, AMI, TTP, DIC, HUS, SLE.

73. A kit comprising:
(a) at least one ADAMTS-13 mutant and/or variant and/or any truncated variant thereof, or any composition thereof, optionally, in a first dosage form, wherein said mutant or any truncated variant thereof, carries at least one mutation and displays resistance and/or reduced sensitivity to cleavage by at least one tPA, or any mutant or variant thereof; and
(b) at least one tPA or any functional fragments or variants thereof, or any composition thereof, optionally, in a second dosage form.

74. The kit according to claim 73, wherein said mutant and/or variant or any truncated variant thereof, carries at least one mutation that substitutes the Arg312 residue and/or any amino acid residue adjacent to said Arg312 of the wild type ADAMTS-13 with a charged amino acid residue, optionally, wherein said charged amino acid residue is any one of lysine, aspartic acid, glutamic acid or histidine.

75. The kit according to claim 73, wherein said mutant, variant, or any truncated variant thereof, carries a mutation substituting the Arginine in position 312 to lysine and is designated R312K, wherein at least one of:
(a) said mutant comprises the amino acid sequence as denoted by SEQ ID NO:11, or any derivatives or variants thereof, and
(b) said truncated variant of said mutant comprises the amino acid sequence as denoted by SEQ ID NO:5, or any derivatives or variants thereof, optionally, wherein said adjacent amino acid reside is valine 313, and optionally, said mutant carries a mutation substituting valine 313 with aspartic acid.

76. The kit according to claim 73, adapted for use in a method for the treatment, amelioration, inhibition or prophylaxis of a disease, disorder, or condition associated with coagulation in a subject in need thereof, optionally, said disease, disorder, or condition is any one of DVT, PE, AIS, AMI, TTP, DIC, HUS, SLE.

77. A mutant of a disintegrin and metalloproteinase with a thrombospondin type 1 motif, member 13 (ADAMTS-13) that carries at least one mutation according to claim 60, wherein said at least one mutation substitutes the Arginine in position 312 (Arg312) of the wild type ADAMTS-13 and/or any amino acid residue adjacent to said Arg312, with a charged amino acid residue, or any truncated variant thereof, or a composition comprising an effective amount of said mutant, said composition optionally further comprises at least one pharmaceutically acceptable carrier, diluent, excipient and/or additive.

78. A combined composition or kit comprising: (a) at least one ADAMTS-13 mutant according to claim 77, or any truncated variant thereof, (b) and at least one tPA or any functional fragments or variants thereof, said composition or kit optionally further comprises at least one pharmaceutically acceptable carrier, diluent, excipient and/or additive.

79. A method for the treatment, amelioration, inhibition or prophylaxis of a disease, disorder, or condition associated with coagulation in a subject in need thereof, the method comprising the step of administering to said subject a therapeutically effective amount of at least one ADAMTS-13 mutant according to claim 77, any truncated variant thereof, or any composition or combined composition comprising said mutant optionally, wherein said subject is a subject treated with at least one tPA, or any mutant or variant thereof, a therapeutically effective amount of at least one ADAMTS-13 mutant, any truncated variant thereof or any composition comprising said mutant.


SEQUENCE LISTING (24 sequences)

SEQ ID NO: 1 - DNA, 4966 bp, Homo sapiens
  misc_feature: ADAMTS13 NM_139025.4
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggtggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtacgcccagcctggtgccccacgaagaagccgctgctccaggacggaccacagccacccctgctggtgcctccctggagtggtcccaggcccggggcctgctcttctccccggctccccagcctcggcggctcctgcccgggccccaggaaaactcagtgcagtccagtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctgagggtcattgaacatttgttccgtgtctggccagccctggagggttgacccctggtctcagtgctttccaattcgaactttttccaatcttaggtatctactttagagtcttctccaatgtccaaaaggctagggggttggaggtggggactctggaaaagcagcccccatttcctcgggtaccaataaataaaacatgcaggctgaccggcgtttttttcttaaaaaaaaaaaaaaaa

SEQ ID NO: 2 - protein, 1427 aa, Homo sapiens
  MISC_FEATURE: ADAMTS NP_620594.1
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 3 - DNA, 2954 bp, Homo sapiens
  misc_feature: tissue plasminogen activator, NM_001319189.1
gaacttcctccccctgctttataaaaacaggcctgcctcagctccctcatggccctgtccactgagcatcctcccgccacacagaaacccgcccagccggggccaccgaccccaccccctgcctggaaacttaaaggaggccggagctgtggggagctcagagctgagatcctacaggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaataggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttccaaaatcagagtggaatggttttgttatagatgctgtatcccactctttattcatgttcacattttaaaatcatttggaattctgcttcactcgcttaacatatacacaacacctgtaacatacaaggcaatgggctaggtgctccagaccgggaaaaggagggacaggaatgcttggtctgatgggctaatatggcatttagagaagtaccaaggtacagtggagccggtcacaaaagggcagacttgtagtagaattcagttgcaagagggattggggaatcttaaggaaaaaatagaatcttaaggaaaaaataactgggtgagacgtggactgtggacaggtgtggaaaaggcactctccatggaggtatgaatatgtagagggccaagagaggggagtacagggagaaatgagttgagcttgtctgaagtgaacttcaggaagaggaacataggctggaatttagattatgggggctctgaacaccaaactgagtttggacttaattgacttctg

SEQ ID NO: 4 - protein, 473 aa, Homo sapiens
  MISC_FEATURE: tissue plasminogen activator, NP_001306118.1
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 5 - protein, 633 aa, Artificial Sequence
  Mutated R312K truncated
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCKVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK

SEQ ID NO: 6 - DNA, 1905 bp, Artificial Sequence
  truncated WT ADAMTS13
agatctgctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggccgactacaaagaccatgacggtgattataaagatcatgatatcgattacaaggatgacgatgacaag

SEQ ID NO: 7 - protein, 635 aa, Artificial Sequence
  truncated WT ADAMTS13
RSAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK

SEQ ID NO: 8 - DNA, 45 bp, Artificial Sequence
  Primers forward to mutate R312 to K
tacagcgccaacgagcagtgccgcgtggccttcggccccaaggct

SEQ ID NO: 9 - DNA, 45 bp, Artificial Sequence
  Primer REVERSE to K
tacagcgccaacgagcagtgcaaggtggccttcggccccaaggct

SEQ ID NO: 10 - DNA, 45 bp, Artificial Sequence
  Primer AntiK
agccttggggccgaaggccaccttgcactgctcgttggcgctgta

SEQ ID NO: 11 - protein, 1427 aa, Artificial Sequence
  ADAMTS-13 R312K
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCKVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 12 - protein, 1427 aa, Artificial Sequence
  ADAMTS-13 R312A
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCAVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 13 - protein, 1427 aa, Artificial Sequence
  ADAMTS-13 V313A
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRAAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 14 - protein, 1427 aa, Artificial Sequence
  ADAMTS-13 V313D
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRDAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 15 - protein, 633 aa, Artificial Sequence
  Mutated R312A truncated
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCAVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK

SEQ ID NO: 16 - protein, 633 aa, Artificial Sequence
  Mutated V313A truncated
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRAAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK

SEQ ID NO: 17 - protein, 633 aa, Artificial Sequence
  Mutated V313D truncated
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRDAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK

SEQ ID NO: 18 - DNA, 1905 bp, Artificial Sequence
  truncated mutated ADAMTS13 R312K
agatctgctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgcaaggtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggccgactacaaagaccatgacggtgattataaagatcatgatatcgattacaaggatgacgatgacaag

SEQ ID NO: 19 - DNA, 1905 bp, Artificial Sequence
  truncated mutated ADAMTS13 R312-A
agatctgctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgcgcggtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggccgactacaaagaccatgacggtgattataaagatcatgatatcgattacaaggatgacgatgacaag

SEQ ID NO: 20 - DNA, 45 bp, Artificial Sequence
  Primers to mutate R312 to A
tacagcgccaacgagcagtgcgcggtggccttcggccccaaggct

SEQ ID NO: 21 - DNA, 1908 bp, Artificial Sequence
  truncated mutated ADAMTS13 V313-D
agatctgctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcaaggtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggccgactacaaagaccatgacggtgattataaagatcatgatatcgattacaaggatgacgatgacaag

SEQ ID NO: 22 - DNA, 1908 bp, Artificial Sequence
  truncated mutated ADAMTS13 V313-A
agatctgctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgcggtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggccgactacaaagaccatgacggtgattataaagatcatgatatcgattacaaggatgacgatgacaag

SEQ ID NO: 23 - protein, 1427 aa, Artificial Sequence
  Double mutant R312K, V313D (full length)
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCKDAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 24 - protein, 633 aa, Artificial Sequence
  Double mutant R312K, V313D truncated
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCKDAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQADYKDHDGDYKDHDIDYKDDDDK